(19) United States
(12) Patent Application Publication
(10) Pub. No.: US 2009/0057613 A1
(43) Pub. Date: Mar. 5, 2009

(54) FLUORESCENT QUINACRIDONES

(75) Inventors: Hiroshi Yamamoto, Hyogo (JP); Norihisa Dan, Kyoto (JP); Paul Adriaan Van Der Schaaf, Hagenthal-le-Haut (FR)

Correspondence Address: JoAnn Villamizar;Ciba Corporation/Patent Department, 540 White Plains Road, P.O. Box 2005, Tarrytown, NY, 10591, US

(73) Assignee: CIBA SPECIALTY CHEMICALS HOLDING INC., Basel (CH)

(21) Appl. No.: 11/630,488

(22) PCT Filed: Jun. 20, 2005
(86) PCT No.: PCT/EP05/52841

§ 371 Date: Sep. 26, 2008

(30) Foreign Application Priority Data
Jun. 29, 2004 (EP) 04103025.5

(51) Int. Cl. C09K 11/06; C07D 471/04; G01N 21/64

(52) U.S. Cl. 252/301.16; 546/56

(57) ABSTRACT

The present invention relates to compounds of the Formula (I), a process for their preparation and their use for the preparation of inks, colorants, pigmented plastics, coatings, non-impact-printing material, color filters, cosmetics, polymeric ink particles, toners, as fluorescent tracers, in color changing media, in solid dye lasers and electroluminescent devices. A luminescent device comprising a composition according to the present invention is high in the efficiency of electrical energy utilisation and high in luminance.

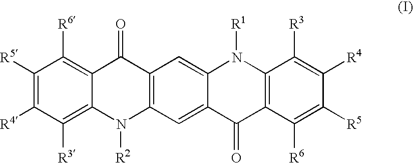

The present invention relates to compounds of the formula

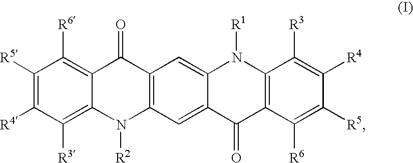

a process for their preparation and their use for the preparation of inks, colorants, pigmented plastics, coatings, non-impact-printing material, color filters, cosmetics, polymeric ink particles, toners, as fluorescent tracers, in color changing media, in solid dye lasers and electroluminescent devices. A luminescent device comprising a composition according to the present invention is high in the efficiency of electrical energy utilisation and high in luminance.

U.S. Pat. No. 6,280,859 relates to a light-emitting material, for example a quinacridone derivative, and an organic EL device for which the light-emitting material is adapted. The following quinacridone derivatives are explicitly mentioned:

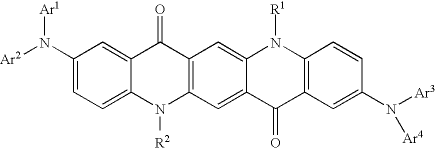

| R¹ = R² | Ar¹ = Ar² = Ar³ = Ar⁴ |
|---|---|
| —C₂H₅ | 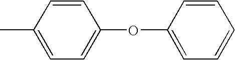 |
| H | 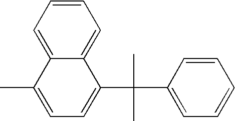 |
| Ph | 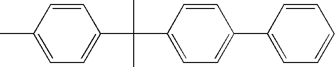 |

EP-A-0939972 relates to an electroluminescent device comprises an electroluminescent element comprising a hole injection and/or hole transport zone containing an optionally substituted tris-1,3,5-(aminophenyl)benzene compound, a luminescent material and a quinacridone derivative. The quinacridone derivative is not a quinacridone substituted by a group —NAr¹Ar².

US200210038867A1 relates to an organic EL device comprising a light emitting layer containing a specific coumarine derivative and a specific quinacridone compound and a hole injecting layer and/or transporting layer containing a specific tetraaryidiamine derivative. The quinacridone compound is not a quinacridone substituted by a group —NAr¹Ar².

Surprisingly, it was found that luminescent devices, which are high in the efficiency of electrical energy utilisation and high in luminance, can be obtained if specific quinacridone compounds or specific combinations of quinacridone and, for example, diketopyrrolopyrrole (DPP) compounds are used, especially as light emitting substances.

Accordingly, the present invention relates to quinacridone compounds of formula

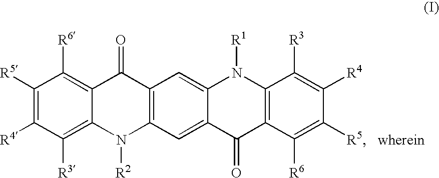

R¹ and R² may be the same or different and are selected from a C₁-C₂₅alkyl group, which can be substituted by fluorine, chlorine or bromine, an allyl group, which can be substituted one to three times with C₁-C₄alkyl, a cycloalkyl group, a cycloalkyl group, which can be condensed one or two times by phenyl which can be substituted one to three times with C₁-C₄-alkyl, halogen, nitro or cyano, an alkenyl group, a cycloalkenyl group, an alkynyl group, a haloalkyl group, a haloalkenyl group, a haloalkynyl group, a ketone or aldehyde group, an ester group, a carbamoyl group, a ketone group, a silyl group, a siloxanyl group, A³ or —CR⁷R⁸—(CH₂)ₘ-A³, wherein

R⁷ and R⁸ independently from each other stand for hydrogen, or C₁-C₄alkyl, or phenyl, which can be substituted one to three times with C₁-C₄alkyl,
A³ stands for aryl or heteroaryl, in particular phenyl or 1- or 2-naphthyl, which can be substituted one to three times with C₁-C₈alkyl and/or C₁-C₈alkoxy, and m stands for 0, 1, 2, 3 or 4,
R³, R^{3′}, R⁶ and R^{6′}, independently of one another, represent hydrogen, halogen, C₁-C₁₈alkyl, halogen-substituted C₁-C₁₈alkyl, C₁-C₁₈alkoxy, C₁-C₁₈₋aalkylthio, cycloalkyl, optionally substituted aryl or arylalkyl, wherein the substituents are alkoxy, halogen or alkyl,
R⁴ and R^{4′} are independently of each other R³, or a group —NAr¹Ar²,
R⁵ and R^{5′} are independently of each other R³, or a group —NAr³Ar⁴, or
R³ and R⁴ and/or R³ and R⁴ together are a group or

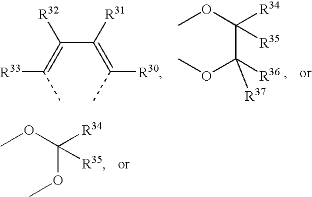

R^{5′} and R^{6′} and/or R⁵ and R⁶ together are a group

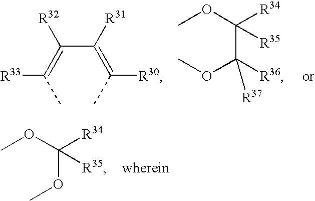

R³⁰, R³¹, R³² and R³³ are independently of each other hydrogen, C₁-C₁₈alkyl, halogen-substituted C₁-C₁₈alkyl, C₁-C₁₈alkoxy, or C₁-C₂₈alkylthio,
R³⁴, RF, R³⁶ and R³⁷ are independently of each other hydrogen, C₁-C₁₈alkyl, halogen-substituted C₁-C₁₈alkyl, C₁-C₁₈alkoxy, or C₁-C₂₈alkylthio,
Ar¹, Ar², Ar³ and Ar⁴ are independently of each other an aryl group, which can optionally be substituted, or a heteroaryl group, which can optionally be substituted, with the proviso that at least one of the groups R⁴, R^{4′}, R⁵ and R^{5′} is a group —NAr¹Ar², or —NAr³Ar⁴, and compounds of formula

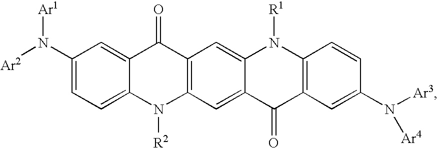

wherein R¹═R²═CH₆ and

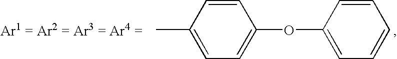

### and R¹═R²=Ph and

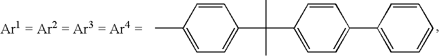

are excluded.

In a preferred embodiment the present invention is directed to compounds of formula

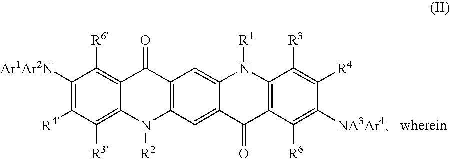

R¹, R², R³, R^{3′}, R⁴, R^{4′}, R⁶, R^{6′}, Ar¹, A² Ar³ and Ar⁴ are as defined above.

Ar¹, Ar², Ar³ and Ar⁴ are preferably independently of each other a group —Ar⁵—X¹—Ar⁶,

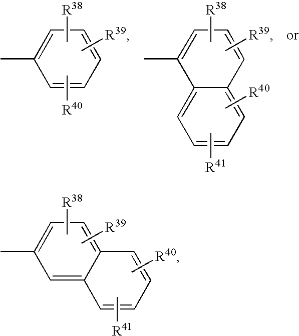

or Ar¹ and Ar² and/or Ar³ and Ar⁴ together with the nitrogen atom to which they are bonded form a five or six membered
heterocyclic ring, such as

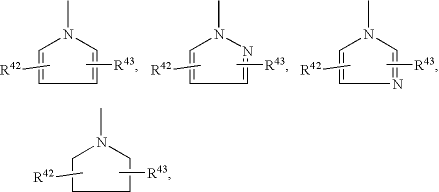

which can be condensed by one or two optionally substituted phenyl groups, such as

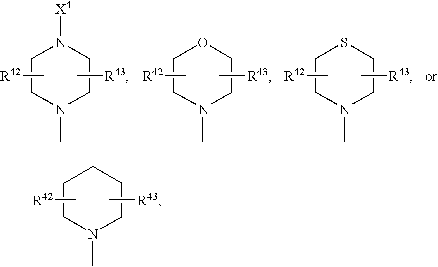

wherein X¹ is C(X²)(X³, —O—, —S—, —SO₂—, —C(═O)—,

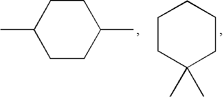

—(CₓH₂ₓ)—O—(C_{y}H_{2y})—, wherein each of x and y is an integer of 0 to 20, while x+y=0 in no case, a substituted or unsubstituted alkylene group having at least two carbon atoms, a substituted or unsubstituted alkylidene group having at least two carbon atoms, a substituted or unsubstituted alicyclic group having at least two carbon atoms,

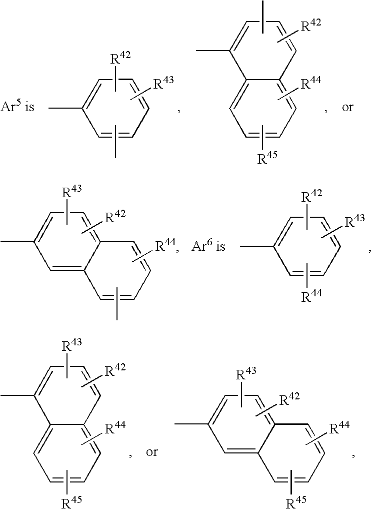

X² and X³ independently from each other stand for hydrogen, C₁-C₁₈alkyl, halogen-substituted C₁-C₁₈alkyl, or phenyl, which can be substituted one to three times with C₁-C₈alkyl and/or C₁-C₈alkoxy,
X⁴ stands for C₁-C₁₈alkyl, halogen-substituted C₁-C₁₈alkyl, or phenyl, which can be substituted one to three times with C₁-C₈alkyl and/or C₁-C₈alkoxy,
R³⁸, R³⁹, R⁴⁰, R⁴¹, R⁴², R⁴³, R⁴ and R⁴⁵ independently from each other stands for hydrogen, C₁-C₈-alkyl, C₁-C₈-alkoxy, or phenyl.

Among the compounds of formula I those are more preferred, wherein two of the groups R⁴, R^{4′}, R⁵ and R^{5′} are a group —NAr¹Ar², or —NAr³Ar⁴, especially R⁴ and R^{4′}, or R⁵ and R^{5′}, wherein those compounds of formula I are especially preferred, wherein R⁵ and R^{5′} are a group —NAr¹Ar², or —NAr³Ar⁴, wherein those compounds of formula I are most preferred, wherein —NAr¹Ar²=—NAr³Ar⁴.

More preferred are quinacridone compounds of formula

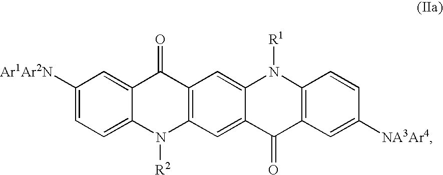

wherein R¹, R², Ar¹, Ar², Ar³ and Ar⁴ are as defined above. The groups —NAr¹Ar², or —NAr³Ar⁴ can be different, but have preferably the same meaning.

In a preferred embodiment of the present invention Ar¹, Ar², Ar³ and Ar⁴ are independently of each other a group

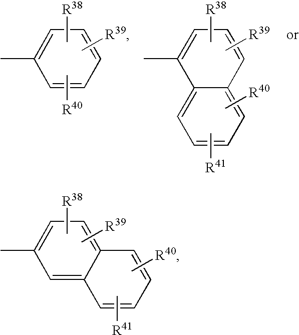

wherein R³⁸, R³⁹, R⁴⁰, and R⁴¹ are as defined above, especially phenyl, tolyl, 2-naphthyl, and 1-naphthyl.

If Ar¹ and Ar² and/or Ar³ and Ar⁴ together with the nitrogen atom to which they are bonded form a five or six membered heterocyclic ring, a group of formula

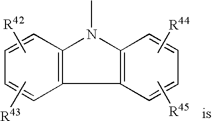

preferred, wherein R⁴², R⁴³, R⁴⁴, and R⁴⁵ are as defined above, especially

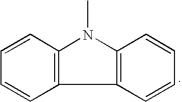

If Ar¹, Ar², Ar³ and Ar⁴ are independently of each other a group —Ar⁵—X¹—Ar⁶, particularly preferred examples of Ar¹, Ar², Ar³ and Ar⁴ are a group

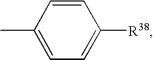

wherein R³⁸ is hydrogen, or C₁-C₄alkyl;

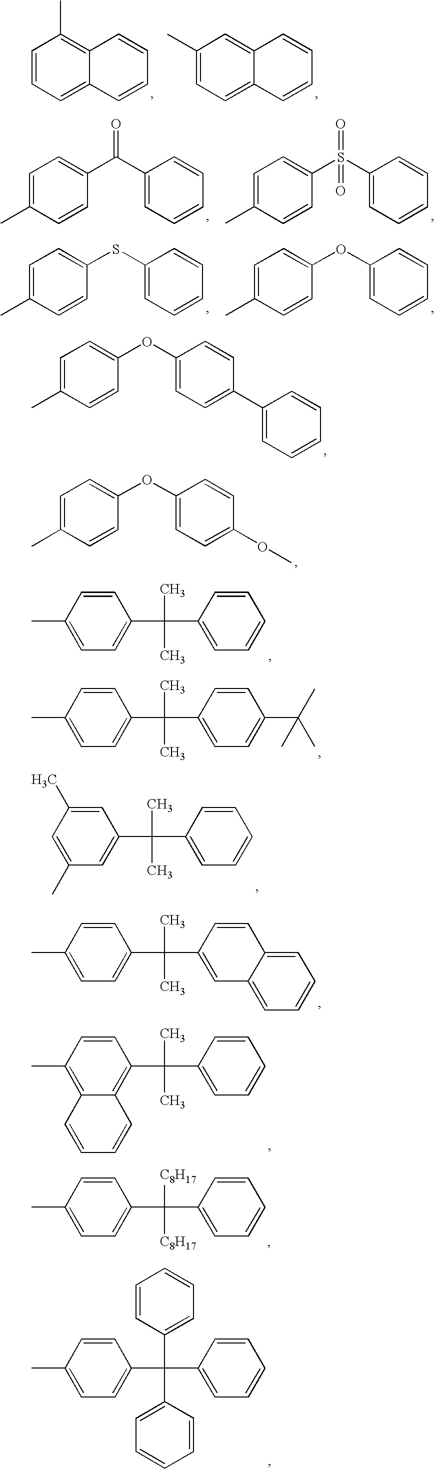

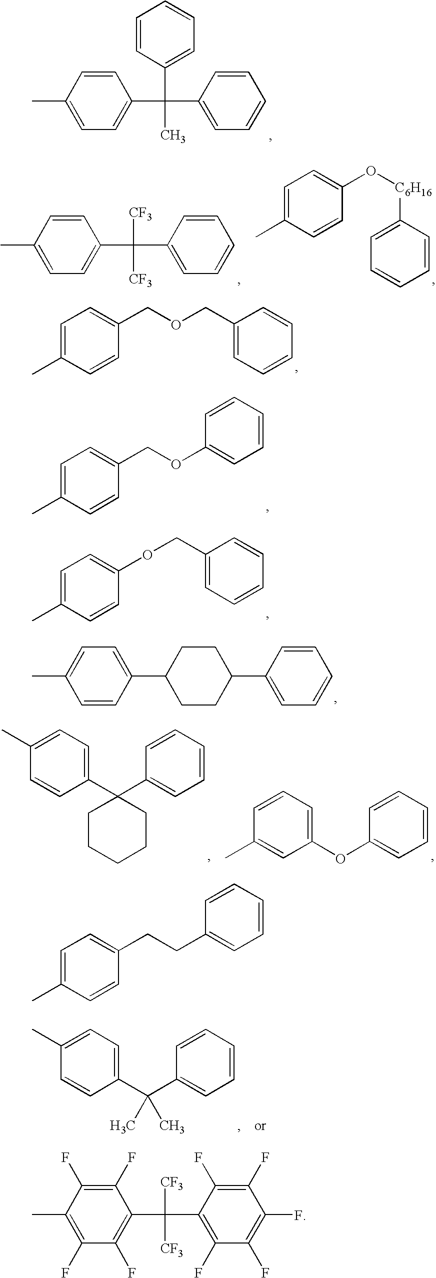

R¹ and R² may be the same or different and are preferably selected from a C₁-C₁₈alkyl group, such as methyl, ethyl, n-propyl, isopropyl, n-butyl, sec.-butyl, isobutyl, tert.-butyl, n-pentyl, 2-pentyl, 3-pentyl, 2,2-dimethylpropyl, n-hexyl, n-heptyl, n-octyl, 1,1,3,3-tetramethylbutyl and 2-ethylhexyl, n-nonyl, decyl, undecyl, dodecyl, tetradecyl, pentadecyl, hexadecyl, heptadecyl, octadecyl; a C₅-C₁₂cycloalkyl group, especially cyclohexyl, which can be substituted one to three times with C₁-C₄-alkyl, or C₁-C₄-alkoxy, or a cycloalkyl group, especially cyclohexyl, which can be condensed one or two times by phenyl which can be substituted one to three times with C₁-C₈-alkyl, C₁-C₈-alkoxy, halogen and cyano, especially

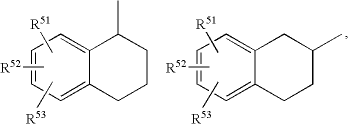

or wherein R⁵¹, R⁵² and R⁵³ are independently of each other hydrogen, C₁-C₈-alkyl, C₁-C₈-alkoxy, halogen and cyano; a C₅-C₁₂cycloalkenyl group, especially cyclohexenyl, which can be substituted one to three times with C₁-C₄-alkyl, or C₁-C₄-alkoxy; a C₆-C₁₄aryl group, especially phenyl, biphenylyl, 1- or 2-naphthyl, which can be substituted one to three times by C₁-C₈alkyl, or C₁-C₈alkoxy; or —CR⁷R⁸—(CH₂)ₘ-A³ wherein

R⁷ and R⁸ stand for hydrogen, or C₁-C₄alkyl, A³ stands for phenyl, 1- or 2-naphthyl, which can be substituted one to three times by C₁-C₈alkyl, or C₁-C₈alkoxy; and m stands for 0, or 1.

More preferably R¹ and R² are independently of each other C₁-C₈alkyl, such as methyl, ethyl, n-propyl, isopropyl, n-butyl, sec.-butyl, isobutyl, tert.-butyl, n-pentyl, 2-pentyl, 3-pentyl, 2,2-dimethylpropyl, n-hexyl, n-heptyl, n-octyl, 1,1,3,3-tetramethylbutyl and 2-ethylhexyl;

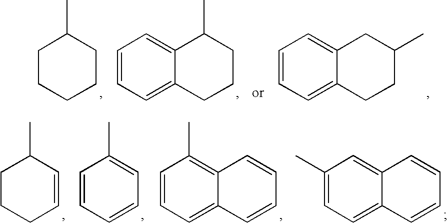

or —CR¹¹R¹²-A⁵, wherein R¹¹ is hydrogen, or methyl, R¹² is hydrogen, or methyl, and A⁵ is

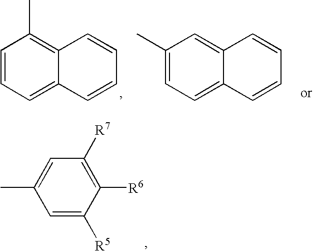

wherein R⁵, R⁶ and R⁷ are independently of each other hydrogen, C₁-C₄-alkyl, phenyl or halogen, wherein groups

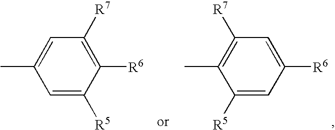

wherein R⁵, R⁶ and R⁷ are hydrogen; R⁶ is C₁-C₄-alkyl, phenyl and R⁵ and R⁷ are hydrogen; R⁵ is C₁-C₄-alkyl and R⁶ and R⁷ are hydrogen; or R⁵ is hydrogen and R⁵ and R⁷ are C₁-C₄-alkyl are most preferred.

The most preferred compounds are listed below:

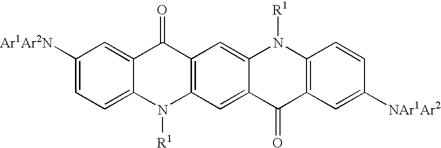

| Compound | R¹ | Ar¹ | Ar² |
|---|---|---|---|
| QA-2 | n-C₆H₁₃ | phenyl | phenyl |
| QA-3 | n-C₆H₁₃ | 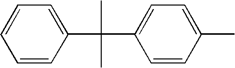 | 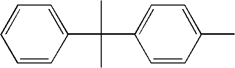 |
| QA-4 | n-C₆H₁₃ | tolyl | tolyl |
| QA-5 | n-C₆H₁₃ | 2-naphthyl | phenyl |
| QA-7 | CH₃ | phenyl | phenyl |
| QA-10 | CH₃ | 2-naphthyl | phenyl |
| QA-11 | C₂H₅ | phenyl | phenyl |
| QA-12 | phenyl | phenyl | phenyl |
| QA-13 | 2-cyclohexene | phenyl | phenyl |
| QA-14 | 2-cyclohexene | 1-naphthyl | phenyl |
| QA-15 | 2-cyclohexene | 4-tolyl | 4-tolyl |
| QA-16 | 2-cyclohexene | 2-naphthyl | phenyl |
| QA-17 | cyclohexane | phenyl | phenyl |
| QA-18 | cyclohexane | 1-naphthyl | phenyl |
| QA-19 | cyclohexane | 4-tolyl | 4-tolyl |
| QA-20 | cyclohexane | 2-naphthyl | phenyl |

A further preferred embodiment of the present invention is directed to compositions comprising a guest chromophore and a host chromophore, wherein the absorption spectrum of the guest chromophore overlaps with the fluorescence emission spectrum of the host chromophore, wherein the host chromophore is a diketopyrrolopyrrole having a photoluminescence emission peak at 500 to 720 nm, preferably 520 to 630 nm, most preferred 540 to 600 nm and wherein the guest chromophore is a compound of formula I.

Such diketopyrrolopyrrole compounds are, for example, described in EP-A-1087005, EP-A-1087006, WO03/002672, WO031022848, WO03064558 and WO2004/090046.

In this embodiment the host chromophore is preferably a diketopyrrolopyrrole (“DPP”) represented by formula

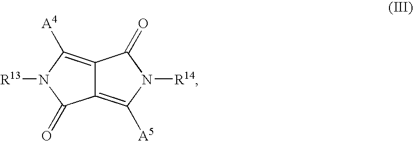

wherein R¹³ and R¹⁴ independently from each other stand for C₁-C₂₅alkyl, which can be substituted by fluorine, chlorine or bromine, C₅-C₁₂-cycloalkyl or C₅-C₁₂cycloalkyl which can be condensed one or two times by phenyl which can be substituted one to three times with C₁-C₄-alkyl, halogen, nitro or cyano, silyl, A⁶ or —CR¹¹R¹²—(CH₂)ₘ-A⁶, wherein R¹¹ and R¹² independently from each other stand for hydrogen, fluorine, chlorine, bromine, cyano or C₁-C₄alkyl, which can be substituted by fluorine, chlorine or bromine, or phenyl which can be substituted one to three times with C₁-C₄alkyl, A⁶ stands for phenyl or 1- or 2-naphthyl which can be substituted one to three times with C₁-C₈alkyl, C₁-C₈alkoxy, halogen, nitro, cyano, phenyl, which can be substituted with C₁-C₈alkyl or C₁-C₈alkoxy one to three times, —NR²³R²⁴, wherein R²³ and R²⁴ represent hydrogen, C₁-C₂₅-alkyl, C₅-C₁₂-cycloalkyl or C₆-C₂₄-aryl, in particular phenyl or 1- or 2-naphthyl which can be substituted one to three times with C₁-C₈alkyl, C₁-C₈alkoxy, halogen or cyano, or phenyl, which can be substituted with C₁-C₈alkyl or C₁-C₈alkoxy one to three times, and m stands for 0, 1, 2, 3 or 4,

A⁴ and A⁵ independently from each other stand for

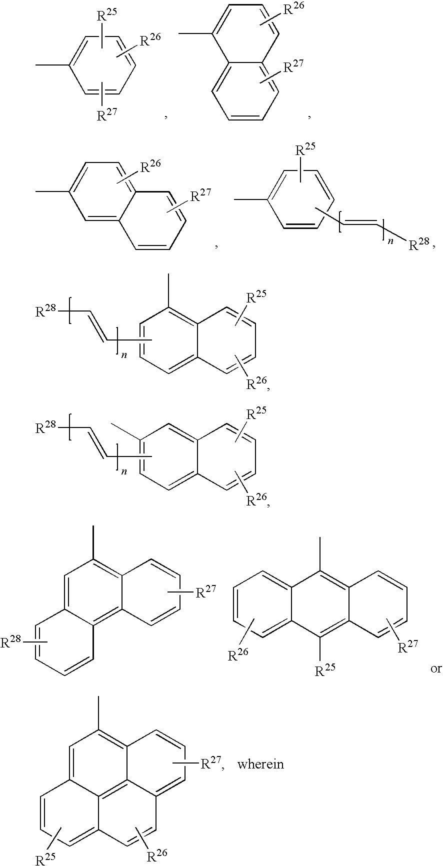

R²⁵, R²⁶, R²⁷ independently from each other stands for hydrogen, C₁-C₂₅-alkyl, —CR¹¹R¹²—(CH₂)ₘ-A⁶, cyano, halogen, —OR²⁹, —S(O)ₚR³⁰, or phenyl, which can be substituted one to three times with C₁-C₈alkyl or C₁-C₈alkoxy, wherein RF stands for C₁-C₂₅-alkyl, C₅-C₁₂-cycloalkyl, —CR¹¹R¹²—(CH₂)ₘ—Ph, C₆-C₂₄-aryl, or a saturated or unsaturated heterocyclic radical comprising five to seven ring atoms, wherein the ring consists of carbon atoms and one to three hetero atoms selected from the group consisting of nitrogen, oxygen and sulfur, R³⁰ stands for C₁-C₂₅-alkyl, C₅-C₁₂-cycloalkyl, —CR¹¹R¹²—(CH₂)ₘ—Ph, R²⁸ stands for C₂-C₂₀-heteroaryl, C₆-C₂₄-aryl, p stands for 0, 1, 2 or 3, m and n stands for 0, 1, 2, 3 or 4.

R¹³ and R¹⁴ independently of each other stand, preferably, for C₁-C₈alkyl, C₅-C₁₂-cycloalkyl, which can be substituted one to three times with C₁-C₈alkyl and/or C₁-C₈alkoxy, phenyl or 1- or 2-naphthyl which can be substituted one to three times with C₁-C₈alkyl and/or C₁-C₈alkoxy, or —CR¹¹R¹²—(CH₂)ₘ-A⁶ wherein R¹¹ and R¹² stand for hydrogen, A⁶ stands for phenyl or 1- or 2-naphthyl, which can be substituted one to three times with C₁-C₈alkyl and/or C₁-C₈alkoxy, and m stands for 0 or 1.

A⁴ and A⁵ independently from each other stand, preferably, for

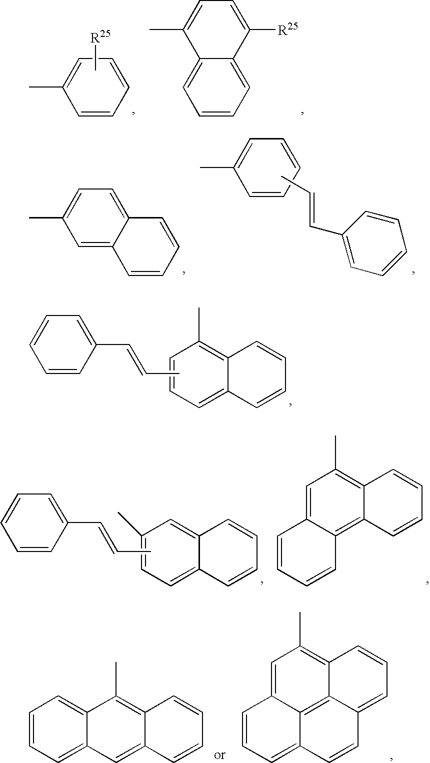

wherein R²⁵ is C₁-C₈-alkyl, phenyl, 1- or 2-naphthyl.

In this embodiment the host chromophore is alternatively a “heterocyclic” diketopyrrolopyrrole (“DPP”) described in WO2004/090046, especially a diketopyrrolopyrrole (“DPP”) represented

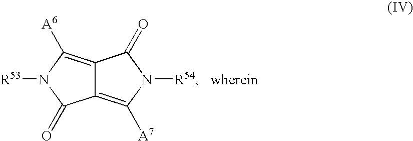

by formula
R⁵³ and R⁵⁴ may be the same or different and are selected from a C₁-C₂₅alkyl group, which can be substituted by fluorine, chlorine or bromine, an allyl group, which can be substituted one to three times with C₁-C₄alkyl, a cycloalkyl group, or a cycloalkyl group, which can be condensed one or two times by phenyl which can be substituted one to three times with C₁-C₄-alkyl, halogen, nitro or cyano, an alkenyl group, a cycloalkenyl group, an alkynyl group, a haloalkyl group, a haloalkenyl group, a haloalkynyl group, a ketone or aldehyde group, an ester group, a carbamoyl group, a ketone group, a silyl group, a siloxanyl group, A⁸ or —CR⁶⁰R⁶¹—(CH₂)ₘ-A⁸, wherein
R⁶⁰ and R⁶¹ independently from each other stand for hydrogen or C₁-C₄alkyl, or phenyl, which can be substituted one to three times with C₁-C₄alkyl,
A⁸ stands for aryl or heteroaryl, in particular phenyl or 1- or 2-naphthyl which can be substituted one to three times with C₁-C₈alkyl and/or C₁-C₈alkoxy, and m stands for 0, 1, 2, 3 or 4,
A⁶ and A⁷ independently from each other are selected from

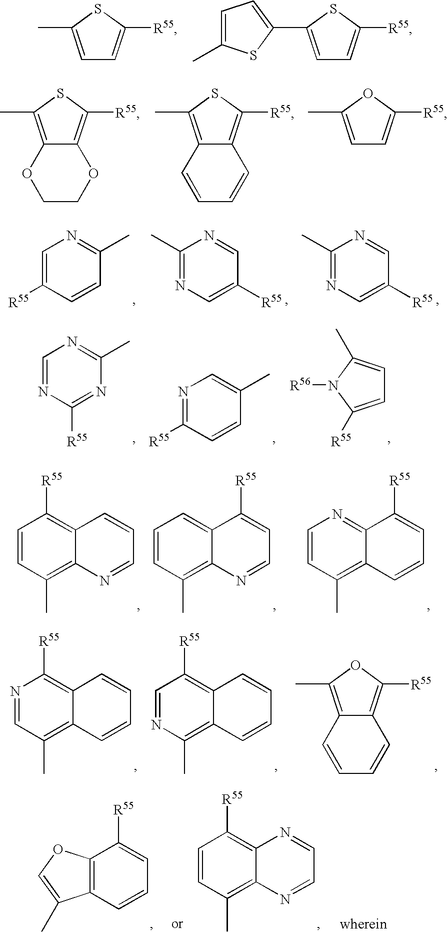

R⁵⁵ is a hydrogen atom, a C₁-C₂alkyl group, a C₁-C₈alkoxy group, a group of formula

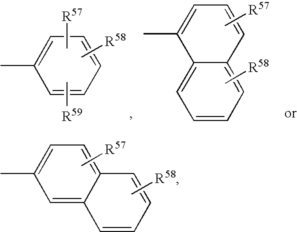

wherein R⁵⁷, R⁵⁸ and R⁵⁹ independently from each other stands for hydrogen, C₁-C₈-alkyl, or C₁₋₈-alkoxy, and R⁵⁶ stands for hydrogen, or C₁-C₈-alkyl.

Preferably R⁵³ and RF independently from each other are selected from C₁-C₁₄alkyl, C₅-C₁₂-cycloalkyl, especially cyclohexyl, which can be substituted one to three times with C₁-C₈alkyl and/or C₁-C₈alkoxy, or C₅-C₁₂-cycloalkyl, especially cyclohexyl, which can be condensed one or two times by phenyl, which can be substituted one to three times with C₁-C₄-alkyl, halogen, nitro or cyano, phenyl or 1- or 2-naphthyl which can be substituted one to three times with C₁-C₈alkyl and/or C₁-C₈alkoxy, or —CR⁶⁰R⁶¹—(CH₂)ₘ-A⁸ wherein R⁶⁰ and R⁶¹ stand for hydrogen, A⁸ stands for phenyl or 1- or 2-naphthyl, which can be substituted one to three times with C₁-C₈alkyl and/or C₁-C₈alkoxy, and m stands for 0 or 1.

Particularly preferred as host chromophores are the DPP compounds represented by the formula III or IV, which are listed below:

| Compound | | |
|---|---|---|
| (of formula | | |
| III) | A⁴ = A⁵ | R¹³ = R¹⁴ |
| H-1 | 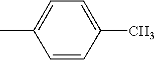 | 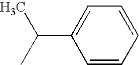 |
| H-2 | 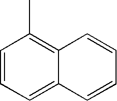 | CH₃, |
| H-3 | 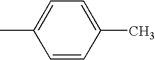 | 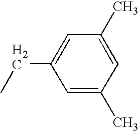 |
| H-4 | 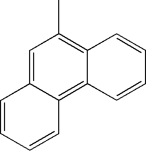 | CH₃, |
| H-5 | 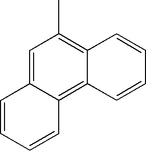 | —CH(CH₃)₂ |
| H-6 | 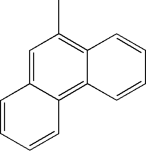 | —(CH₂)₃CH₃ |
| H-7 | 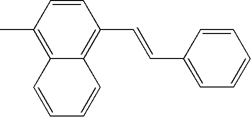 | 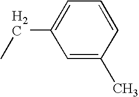 |
| H-8 | 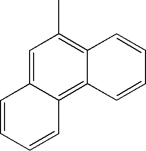 | —Si(CH₃)₃ |
| H-9 | 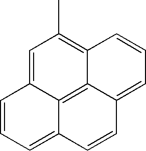 | 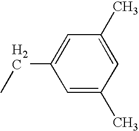 |
| H-10 | 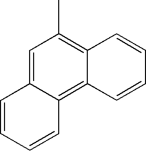 | 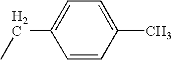 |
| H-11 | 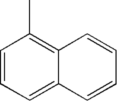 | 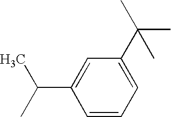 |
| H-12 | 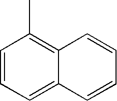 | 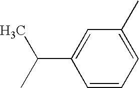 |
| H-13 | 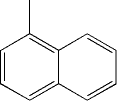 | —CH(CH₃)₂ |
| H-15 | 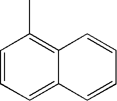 | 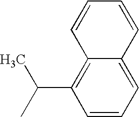 |
| H-16 | 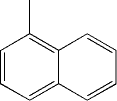 | 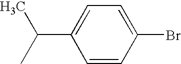 |
| H-16 | 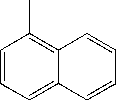 | 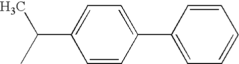 |
| H-17 | 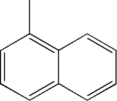 | —CH(CH₃)₂ |
| H-18 | 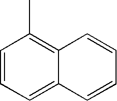 | 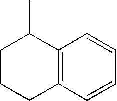 |
| H-19 | 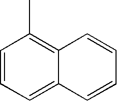 | 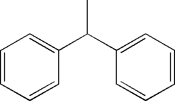 |
| H-20 | 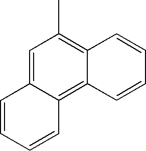 | 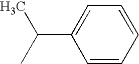 |
| H-21 | 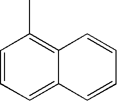 | 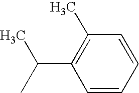 |
| H-22 | 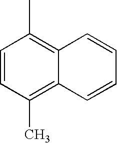 | —CH₃ |
| H-23 | 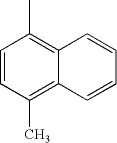 | —CH(CH₃)₂ |
| H-24 | 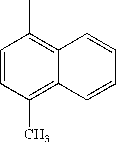 | 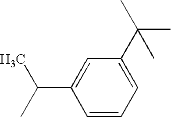 |
| H-25 | 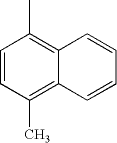 | n-C₁₂H₂₅ |
| H-26 | | —CH₂F |
| H-27 | 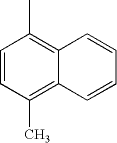 | 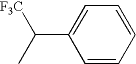 |
| H-28 | 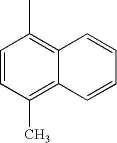 | 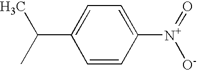 |
| H-29 | 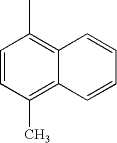 | 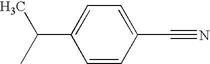 |
| Compound | | |
| (of formula | | |
| IV) | A⁶ = A⁷ | R⁵³ = R⁵⁴ |
| H-30 | 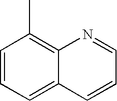 | —CH₃ |
| H-31 | 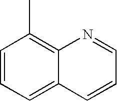 | —CH(CH₃)₂ |
| H-32 | 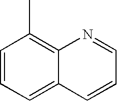 | 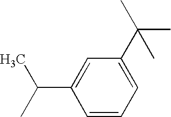 |
| H-33 | 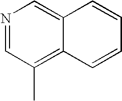 | —CH₃ |
| H-34 | 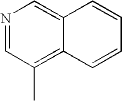 | —CH(CH₃)₂ |
| H-35 | 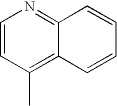 | —CH₃ |
| H-36 | 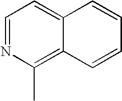 | —CH₃ |
| H-37 | 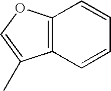 | —CH(CH₃)₂ |
| H-38 | 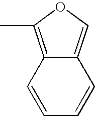 | —CH₃ |
| H-39 | 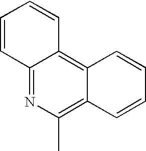 | —CH₃ |
| H-40 | 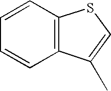 | —CH(CH₃)₂ |
| H-41 | 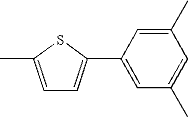 | —CH₃ |
| H-42 | 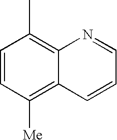 | —CH₃ |
| H-43 | 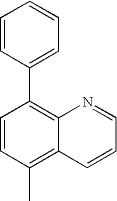 | —CH₃ |
| H-44 | 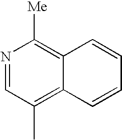 | —CH₃ |
| H-45 | 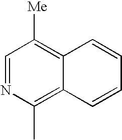 | —CH₃ |
| H-46 | 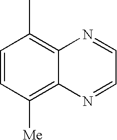 | —CH₃ |
| H-47 | 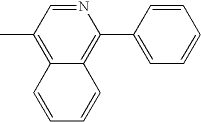 | —CH₃ |

The weight ratio of the host chromophore to the guest chromophore is in general 50:50 to 99.99:0.01, preferably 90:10 to 99.99:0.01, more preferably 95:5 to 99.9:0.1, most preferred 98:2 to 99.9:0.1.

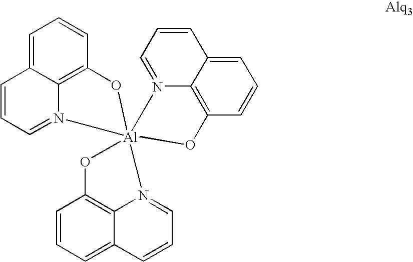

Particularly preferred inventive host/guest compositions comprise and the derivatives thereof, Znq₂, Zn(OX)₂, Zn(BTZ)₂, BeBq₂, Be(5Fla)₂, Balq₂, AJPh₃, Zn(ODZ)₂, Zn(TDZ)₂, Zn(PhPy)₂, Zn(BIZ), Alpq₃, Al(ODZ)₃, Zn(NOD)₂, Zn(Phq)₂, or Zn(NOOD)₂ as host and the quinacridone compounds of formula (I) as guest.

In addition, the host/guest compositions can be optionally used with other known fluorescent compounds as an additional dopant, for example, fused derivatives of aromatic hydrocarbons such as rubrene and perylene; fused heterocyclics such as pyridinothiadiasole, pyrazolopyridine and naphtalimide derivatives; rare earth complexes, such as Eu, Ir, or Pt complexes; zincporphyrin, rhodamine, deazaflavin derivatives, coumarine derivatives, phenoxazones, quinacridones, dicyanoethenylarenes, or the pyrromethene metal complexes disclosed in EP-A-1,253,151, JP2001 257077, JP2001 257078, and JP2001 297881. Compounds of formula I can be prepared by a process, which comprises reacting a quinacridone compound

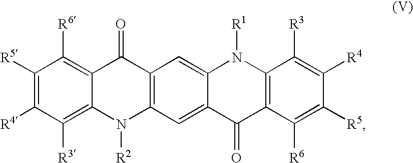

wherein at least one of the groups R⁴, R^{4′}, R⁵ and R⁶ is halogen, preferably Cl, or Br, with a nucleophilic agent HNAr¹Ar² in the presence of an (anhydrous) organic solvent, such as, for example o-xylene, and of an (anhydrous) base, such as, for example, sodium tert-butoxide, at a temperature in the range of from usually 100 to 220° C. optionally in the presence of a catalyst as described, for example, in WO99/47474, such as, for example, [(allyl)PdBr(P(iPr)₃)].

The compounds of formula V can be prepared by reacting compounds of formula

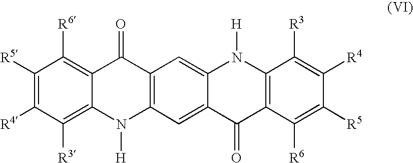

with a halogen compound R¹—X, wherein at least one of the groups R⁴, R^{4′}, R⁵ and R^{5′} is halogen, preferably 1, or Br, in the presence of a base, such as, for example, sodium hydride, in an organic solvent, such as, for example, dry N-methylpyrrolidone (NMP). The compounds of formula VI are commercially available, such as, for example C. I. Pigment Red 202, or C.I. Pigment Red 209, or can be prepared according to or in analogy to procedures known in the state of the art, see, for example EP-A-933972.

The term “halogen” means fluorine, chlorine, bromine and iodine.

C₁-C₂₅alkyl is typically linear or branched—where possible—methyl, ethyl, n-propyl, isopropyl, n-butyl, sec.-butyl, isobutyl, tert.-butyl, n-pentyl, 2-pentyl, 3-pentyl, 2,2-dimethylpropyl, n-hexyl, n-heptyl, n-octyl, 1,1,3,3-tetramethylbutyl and 2-ethylhexyl, n-nonyl, decyl, undecyl, dodecyl, tetradecyl, pentadecyl, hexadecyl, heptadecyl, octadecyl, eicosyl, heneicosyl, docosyl, tetracosyl or pentacosyl, preferably C₁-C₈alkyl such as methyl, ethyl, n-propyl, isopropyl, n-butyl, sec.-butyl, isobutyl, tert.-butyl, n-pentyl, 2-pentyl, 3-pentyl, 2,2-dimethyl-propyl, n-hexyl, n-heptyl, n-octyl, 1,1,3,3-tetramethylbutyl and 2-ethylhexyl, more preferably C₁-C₄alkyl such as typically methyl, ethyl, n-propyl, isopropyl, n-butyl, sec.-butyl, isobutyl, tert.-butyl.

The terms “haloalkyl (or halogen-substituted alkyl), haloalkenyl and haloalkynyl” mean groups given by partially or wholly substituting the above-mentioned alkyl group, alkenyl group and alkynyl group with halogen, such as trifluoromethyl etc. The “aldehyde group, ketone group, ester group, carbamoyl group and amino group” include those substituted by an alkyl group, a cycloalkyl group, an aryl group, an aralkyl group or a heterocyclic group, wherein the alkyl group, the cycloalkyl group, the aryl group, the aralkyl group and the heterocyclic group may be unsubstituted or substituted. The term “silyl group” means a group of formula —SiR⁶²R⁶³R⁶⁴, wherein R⁶², R⁶³ and R⁶⁴ are independently of each other a C₁-C₈alkyl group, in particular a C₁-C₄ alkyl group, a C₆-C₂₄aryl group or a C₇-C₁₂aralkylgroup, such as a trimethylsilyl group. The term “siloxanyl group” means a group of formula —O—SiR⁶²R⁶³R⁶⁴, wherein R², R⁶³ and R⁶⁴ are as defined above, such as a trimethylsiloxanyl group.

Examples of C₁-C₈alkoxy are methoxy, ethoxy, n-propoxy, isopropoxy, n-butoxy, sec.-butoxy, isobutoxy, tart.-butoxy, n-pentoxy, 2-pentoxy, 3-pentoxy, 2,2-dimethylpropoxy, n-hexoxy, n-heptoxy, n-octoxy, 1,1,3,3-tetramethylbutoxy and 2-ethylhexoxy, preferably C₁-C₄alkoxy such as typically methoxy, ethoxy, n-propoxy, isopropoxy, n-butoxy, sec.-butoxy, isobutoxy, tert.-butoxy. The term “alkylthio group” means the same groups as the alkoxy groups, except that the oxygen atom of ether linkage is replaced by a sulfur atom.

The term “aryl group” is typically C₈-C₂₄aryl, such as phenyl, pentalenyl, indenyl, azulenyl, 1-naphthyl, 2-naphthyl, 4-biphenylyl, as-indacenyl, s-indacenyl, acenaphthylenyl, phenanthryl, terphenyl, pyrenyl, 2- or 9-fluorenyl, fluoranthenyl, acephenanthrylenyl, aceanthrylenyl, triphenylenyl, pyrenyl, or anthracenyl, preferably C₆-C₁₂aryl such as phenyl, 1-naphthyl, 2-naphthyl, 4-biphenyl, which may be unsubstituted or substituted.

The term “aralkyl group” is typically C₇-C₂₄aralkyl, such as benzyl, 2-benzyl-2-propyl, β-phenyl-ethyl, α,α-dimethylbenzyl, ω-phenyl-butyl, ω,ω-dimethyl-ωphenyl-butyl, ω-phenyl-dodecyl, ω-phenyl-octadecyl, ω-phenyl-eicosyl or ω-phenyl-docosyl, preferably C₇-C₁₈aralkyl such as benzyl, 2-benzyl-2-propyl, β-phenyl-ethyl, α,α-dimethylbenzyl, ω-phenyl-butyl, ω,ω-dimethyl-phenyl-butyl, ω-phenyl-dodecyl or ω-phenyl-octadecyl, and particularly preferred C₇-C₁₂aralkyl such as benzyl, 2-benzyl-2-propyl, β-phenyl-ethyl, α,α-dimethylbenzyl, ω-phenyl-butyl, or ω,ω-dimethyl-ω-phenyl-butyl, in which both the aliphatic hydrocarbon group and aromatic hydrocarbon group may be unsubstituted or substituted.

The term “aryl ether group” is typically a C₆₋₂₄aryloxy group, that is to say O—C₆₋₂₄aryl, such as, for example, phenoxy or 4-methoxyphenyl. The term “aryl thioether group” is typically a C₆₋₂₄arylthio group, that is to say S—C₆₋₂₄aryl, such as, for example, phenylthio or 4-methoxyphenylthio. The term “carbamoyl group” is typically a C₁₋₁₈carbamoyl radical, preferably C₁₋₈carbamoyl radical, which may be unsubstituted or substituted, such as, for example, carbamoyl, methylcarbamoyl, ethylcarbamoyl, n-butylcarbamoyl, tert-butylcarbamoyl, dimethylcarbamoyloxy, morpholinocarbamoyl or pyrrolidinocarbamoyl.

The term “cycloalkyl group” is typically C₅-C₁₂cycloalkyl, such as cyclopentyl, cyclohexyl, cycloheptyl, cyclooctyl, cyclononyl, cyclodecyl, cycloundecyl, cyclododecyl, preferably cyclopentyl, cyclohexyl, cycloheptyl, or cyclooctyl, which may be unsubstituted or substituted.

The term “cycloalkenyl group” means an unsaturated alicyclic hydrocarbon group containing one or more double bonds, such as cyclopentenyl, cyclopentadienyl, cyclohexenyl and the like, which may be unsubstituted or substituted. The cycloalkyl group, in particular a cyclohexyl group, can be condensed one or two times by phenyl which can be substituted one to three times with C₁-C₄-alkyl, halogen and cyano. Examples of such condensed cyclohexyl groups are:

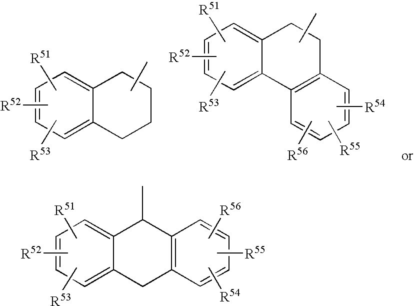

in particular

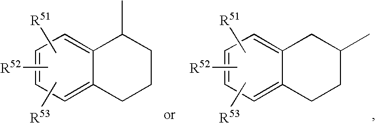

wherein R⁵¹, R⁵², R⁵³, R⁵⁴, R⁵⁵ and R⁵⁸ are independently of each other C₁-C₈-alkyl, C₁-C₈-alkoxy, halogen and cyano, in particular hydrogen.

The term “heteroaryl or heterocyclic group” is a ring with five to seven ring atoms, wherein nitrogen, oxygen or sulfur are the possible hetero atoms, and is typically an unsaturated heterocyclic radical with five to 18 atoms having at least six conjugated π-electrons such as thienyl, benzo[b]thienyl, dibenzo[b,d]thienyl, thianthrenyl, furyl, furfuryl, 2H-pyranyl, benzofuranyl, isobenzofuranyl, dibenzofuranyl, phenoxythienyl, pyrrolyl, imidazolyl, pyrazolyl, pyridyl, bipyridyl, triazinyl, pyrimidinyl, pyrazinyl, pyridazinyl, indolizinyl, isoindolyl, indolyl, indazolyl, purinyl, quinolizinyl, chinolyl, isochinolyl, phthalazinyl, naphthyridinyl, chinoxalinyl, chinazolinyl, cinnolinyl, pteridinyl, carbazolyl, carbolinyl, benzotriazolyl, benzoxazolyl, phenanthridinyl, acridinyl, perimidinyl, phenanthrolinyl, phenazinyl, isothiazolyl, phenothiazinyl, isoxazolyl, furazanyl or phenoxazinyl, preferably the above-mentioned mono- or bicyclic heterocyclic radicals.

The terms “aryl” and “alkyl” in alkylamino groups, dialkylamino groups, alkylarylamino groups, arylamino groups and diarylgroups are typically C₁-C₂₅alkyl and C₆-C₂₄aryl, respectively.

The above-mentioned groups can be substituted by a C₁-C₈alkyl, a hydroxyl group, a mercapto group, C₁-C₈alkoxy, C₁-C₈alkylthio, halogen, halo-C₁-C₈alkyl, a cyano group, an aldehyde group, a ketone group, a carboxyl group, an ester group, a carbamoyl group, an amino group, a nitro group, a silyl group, or a siloxanyl group.

The present invention relates further to an electroluminescent device having the compounds of formula I or the compositions according to the present invention between an anode and a cathode and emitting light by the action of electrical energy.

Typical constitutions of latest organic electroluminescent devices are:

(i) an anode/a hole transporting layer/an electron transporting layer/a cathode, in which the compounds or compositions of the present invention are used either as positive-hole transport compound or composition, which is exploited to form the light emitting and hole transporting layers, or as electron transport compounds or compositions, which can be exploited to form the light-emitting and electron transporting layers,
(ii) an anode/a hole transporting layer/a light-emitting layer/an electron transporting layer/a cathode, in which the compounds or compositions form the light-emitting layer regardless of whether they exhibit positive-hole or electron transport properties in this constitution,
(iii) an anode/a hole injection layer/a hole transporting layer/a light-emitting layer/an electron transporting layer/a cathode,
(iv) an anode/a hole transporting layer/a light-emitting layer/a positive hole inhibiting layer/an electron transporting layer/a cathode,
(v) an anode/a hole injection layer/a hole transporting layer/a light-emitting layer/a positive hole inhibiting layer/an electron transporting layer/a cathode,
(vi) an anode/a light-emitting layer/an electron transporting layer/a cathode,
(vii) an anode/a light-emitting layer/a positive hole inhibiting layer/an electron transporting layer/a cathode,
(viii) a mono-layer containing a light emitting material alone or a combination a light emitting material and any of materials of the hole transporting layer, the hole-blocking layer and/or the electron transporting layer, and
(ix) a multi-layered structure described in (ii) to (vii), wherein a light emitting layer is the mono-layer defined in (viii).

The compounds and compositions of the present invention can, in principal, be used for any organic layer, such as, for example, hole transporting layer, light emitting layer, or electron transporting layer, but are preferably used as the light emitting material in the light emitting layer.

Thin film type electroluminescent devices usually consist essentially of a pair of electrodes and at least one charge transporting layer in between. Usually two charge transporting layers, a hole transporting layer (next to the anode) and an electron transporting layer (next to the cathode) are present Either one of them contains—depending on its properties as hole-transporting or electron-transporting material—an inorganic or organic fluorescence substance as light-emitting material. It is also common, that a light-emitting material is used as an additional layer between the hole-transporting and the electron-transporting layer. In the above mentioned device structure, a hole injection layer can be constructed between an anode and a hole transporting layer and/or a positive hole inhibiting layer can be constructed between a light emitting layer and an electron transporting layer to maximise hole and electron population in the light emitting layer, reaching large efficiency in charge recombination and intensive light emission.

The devices can be prepared in several ways. Usually, vacuum evaporation is used for the preparation. Preferably, the organic layers are laminated in the above order on a commercially available indium-tin-oxide (“ITO”) glass substrate held at room temperature, which works as the anode in the above constitutions. The membrane thickness is preferably in the range of 1 to 10,000 nm, more preferably 1 to 5,000 nm, more preferably 1 to 1,000 nm, more preferably 1 to 500 nm. The cathode metal, such as a Mg/Ag alloy, a binary Li—Al or LiF—Al system with an thickness in the range of 50-200 nm is laminated on the top of the organic layers. The vacuum during the deposition is preferably less than 0.1333 Pa (1×10⁻³ Torr), more preferably less than 1.333×10⁻³ Pa (1×10⁻⁻⁵ Torr), more preferably less than 1.333×10⁻⁴ Pa (1×10⁻⁷ Torr).

As anode usual anode materials which possess high work function such as metals like gold, silver, copper, aluminum, indium, iron, zinc, tin, chromium, titanium, vanadium, cobalt, nickel, lead, manganese, tungsten and the like, metallic alloys such as magnesium/copper, magnesium/silver, magnesium/aluminum, aluminum/indium and the like, semiconductors such as Si, Ge, GaAs and the like, metallic oxides such as indium-tin-oxide (“ITO”), ZnO and the like, metallic compounds, such as CuI and the like, and furthermore, electroconducting polymers, such as polyacetylene, polyaniline, polythiophene, polypyrrole, polyparaphenylene and the like, preferably ITO, most preferably ITO on glass as substrate can be used. Of these electrode materials, metals, metallic alloys, metallic oxides and metallic compounds can be transformed into electrodes, for example, by means of the sputtering method. In the case of using a metal or a metallic alloy as a material for an electrode, the electrode can be formed also by the vacuum deposition method. In the case of using a metal or a metallic alloy as a material forming an electrode, the electrode can be formed, furthermore, by the chemical plating method (see for example, Handbook of Electrochemistry, pp 383-387, Mazuren, 1985). In the case of using an electroconducting polymer, an electrode can be made by forming it into a film by means of anodic oxidation polymerization method onto a substrate which is previously provided with an electroconducting coating. The thickness of an electrode to be formed on a substrate is not limited to a particular value, but, when the substrate is used as a light emitting plane, the thickness of the electrode is preferably within the range of from 1 nm to 300 nm, more preferably, within the range of from 5 to 200 nm so as to ensure transparency.

In a preferred embodiment ITO is used on a substrate having an ITO film thickness in the range of from 10 nm (100 Å) to 1μ (10000 Å), preferably from 20 nm (200 Å) to 500 nm (5000 Å). Generally, the sheet resistance of the ITO film is chosen in the range of not more than 100 Ω/cm², preferably not more than 50 Ω/cm².

Such anodes are commercially available from Japanese manufacturers, such as Geomatech Co. Ltd., Sanyo Vacuum Co. Ltd., Nippon Sheet Glass Co. Ltd.

As substrate either an electronconducting or electrically insulating material can be used. In case of using an electroconducting substrate, a light emitting layer or a positive hole transporting layer is directly formed thereupon, while in case of using an electrically insulating substrate, an electrode is firstly formed thereupon and then a light emitting layer or a positive hole transporting layer is superposed.

The substrate may be either transparent, semi-transparent or opaque. However, in case of using a substrate as an indicating plane, the substrate must be transparent or semi-transparent.

Transparent electrically insulating substrates are, for example, inorganic compounds such as glass, quartz and the like, organic polymeric compounds such as polyethylene, polypropylene, polymethylmethacrylate, polyacrylonitrile, polyester, polycarbonate, polyvinylchloride, polyvinylalcohol, polyvinylacetate and the like. Each of these substrates can be transformed into a transparent electroconducting substrate by providing it with an electrode according to one of the methods described above.

Examples of semi-transparent electrically insulating substrates are inorganic compounds such as alumina, YSZ (yttrium stabilized zirconia) and the like, organic polymeric compounds such as polyethylene, polypropylene, polystyrene, epoxy resins and the like. Each of these substrates can be transformed into a semitransparent electroconducting substrate by providing it with an electrode according to one of the above-mentioned methods.

Examples of opaque electroconducting substrates are metals such as aluminum, indium, iron, nickel, zinc, tin, chromium, titanium, copper, silver, gold, platinum and the like, various electroplated metals, metallic alloys such as bronze, stainless steel and the like, semiconductors such as Si, Go, GaAs, and the like, electroconducting polymers such as polyaniline, polythiophene, polypyrrole, polyacetylene, polyparaphenylene and the like.

A substrate can be obtained by forming one of the above listed substrate materials to a desired dimension. It is preferred that the substrate has a smooth surface. Even, if it has a rough surface, it will not cause any problem for practical use, provided that it has round unevenness having a curvature of not less than 20 μm. As for the thickness of the substrate, there is no restriction as far as it ensures sufficient mechanical strength.

As cathode usual cathode materials which possess low work function such as alkali metals, earth alkaline metals, group 13 elements, silver, and copper as well as alloys or mixtures thereof such as sodium, lithium, potassium, calcium, lithium fluoride (LiF), sodium-potassium alloy, magnesium, magnesium-silver alloy, magnesium-copper alloy, magnesium-aluminum alloy, magnesium-indium alloy, aluminum, aluminum-aluminum oxide alloy, aluminum-lithium alloy, indium, calcium, and materials exemplified in EP-A 499,011 such as electroconducting polymers e.g. polypyrrole, polythiophene, polyaniline, polyacetylene etc., preferably Mg/Ag alloys, LiF—Al or Li—Al compositions can be used.

In a preferred embodiment a magnesium-silver alloy or a mixture of magnesium and silver, or a lithium-aluminum alloy, lithium fluoride-aluminum alloy or a mixture of lithium and aluminum can be used in a film thickness in the range of from 10 nm (100 Å) to 1 μm (10000 Å), preferably from 20 nm (200 Å) to 500 nm (5000 Å).

Such cathodes can be deposited on the foregoing electron transporting layer by known vacuum deposition techniques described above.

In a preferred embodiment of this invention a light-emitting layer can be used between the hole transporting layer and the electron transporting layer. Usually the light-emitting layer is prepared by forming a thin film on the hole transporting layer.

As methods for forming said thin film, there are, for example, the vacuum deposition method, the spin-coating method, the casting method, the Langmuir-Blodgett (“LB”) method and the like. Among these methods, the vacuum deposition method, the spin-coating method and the casting method are particularly preferred in view of ease of operation and cost.

In case of forming a thin film using a composition by means of the vacuum deposition method, the conditions under which the vacuum deposition is carried out are usually strongly dependent on the properties, shape and crystalline state of the compound(s). However, optimum conditions are usually as follows: temperature of the heating boat: 100 to 400° C.; substrate temperature: —100 to 350° C.; pressure:1.33×10⁴ Pa (1×10² Torr) to 1.33×10⁴ Pa (1×10⁻⁶ Torr) and deposition rate: 1 pm to 6 nm/sec.

In an organic EL element, the thickness of the light emitting layer is one of the factors determining its light emission properties. For example, if a light emitting layer is not sufficiently thick, a short circuit can occur quite easily between two electrodes sandwiching said light emitting layer, and therefor, no EL emission is obtained. On the other hand, if the light emitting layer is excessively thick, a large potential drop occurs inside the light emitting layer because of its high electrical resistance, so that the threshold voltage for EL emission increases. Accordingly, the thickness of the organic light emitting layer is limited to the range of from 5 nm to 5 μm, preferably to the range of from 10 nm to 500 nm.

In the case of forming a light emitting layer by using the spin-coating method and the casting method, Ink jet printing method, the coating can be carried out using a solution prepared by dissolving the composition in a concentration of from 0.0001 to 90% by weight in an appropriate organic solvent such as benzene, toluene, xylene, tetrahydrofurane, methyltetrahydrofurane, N,N-dimethylformamide, dichloromethane, dimethylsulfoxide and the like. If the concentration exceeds 90% by weight, the solution usually is so viscous that it no longer permits forming a smooth and homogenous film. On the other hand, if the concentration is less than 0.0001% by weight, the efficiency of forming a film is too low to be economical. Accordingly, a preferred concentration of the composition is within the range of from 0.01 to 80% by weight.

In the case of using the above spin-coating or casting method, it is possible to further improve the homogeneity and mechanical strength of the resulting layer by adding a polymer binder to the solution for forming the light emitting layer. In principle, any polymer binder may be used, provided that it is soluble in the solvent in which the composition is dissolved. Examples of such polymer binders are polycarbonate, polyvinylalcohol, polymethacrylate, polymethylmethacrylate, polyester, polyvinylacetate, epoxy resin and the like. However, if the solid content composed of the polymer binder and the composition exceeds 99% by weight, the fluidity of the solution is usually so low that it is impossible to form a light emitting layer excellent in homogeneity. On the other hand, if the content of the composition is substantially smaller than that of the polymer binder, the electrical resistance of said layer is very large, so that it does not emit light unless a high voltage is applied thereto. Accordingly, the preferred ratio of the polymer binder to the composition is chosen within the range of from 10:1 to 1:50 by weight, and the solid content composed of both components in the solution is preferably within the range of from 0.01 to 80% by weight, and more preferably, within the range of 0.1 to 60% by weight.

As hole-transporting layers known organic hole transporting compounds such as polyvinyl carbazole

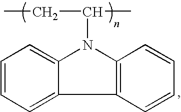

a TPD compound disclosed in J. Amer. Chem. Soc. 90 (1968) 3925:

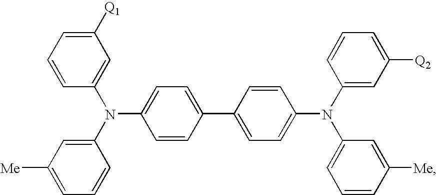

wherein Q₁ and Q₂ each represent a hydrogen atom or a methyl group; a compound disclosed in J. Appl. Phys. 65(9) (1989) 3610:

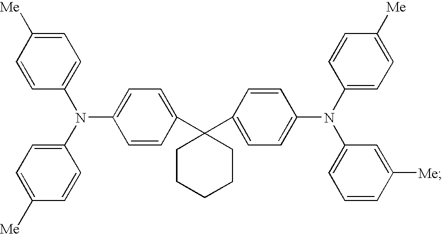

a stilbene based compound

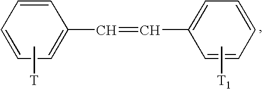

wherein T and T₁ stand for an organic radical;

a hydrazone based compound

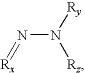

wherein Rₓ, R_{y} and R_{z} stand for an organic radical, and the like can be used.

Compounds to be used as a positive hole transporting material are not restricted to the above listed compounds. Any compound having a property of transporting positive holes can be used as a positive hole transporting material such as triazole derivatives, oxadiazole derivatives, imidazole derivatives, polyarylalkane derivatives, pyrazoline derivative, pyrazolone derivatives, phenylene diamine derivatives, arylamine derivatives, amino substituted chalcone derivatives, oxazole derivatives, stilbenzylanthracene derivatives, fluorenone derivatives, hydrazone derivatives, stilbene derivatives, copolymers of aniline derivatives, PEDOT (poly (3,4-ethylenedioxy-thiophene)) and the derivatives thereof, electro-conductive oligomers, particularly thiophene oligomers, porphyrin compounds, aromatic tertiary amine compounds, stilbenzyl amine compounds etc.

Particularly, aromatic tertiary amine compounds such as N,N,N′,N′-tetraphenyl-4,4′-diaminobiphenyl, N,N-diphenyl-N,N′-bis(3-methylphenyl)-4,4′-diaminobiphenyl (TPD), 2,2′-bis(di-p-torylaminophenyl)propane, 1,1′-bis(4-di-torylaminophenyl)-4-phenylcyclohexane, bis(4-dimethylamino-2-methylphenyl)phenylmethane, bis(4-di-p-tolylaminophenyl)phenyl-methane, N,N′-diphenyl-N,N′-di(4-methoxyphenyl)-4,4′-diaminobiphenyl, N,N,N′,N′-tetraphenyl-4,4′-diaminodiphenylether, 4,4′-bis(diphenylamino)quaterphenyl, N,N,N-tri(p-tolyl)amine, 4-(di-p-tolylamino)-4′-[4-(di-p-tolylamino)stilyl]stilbene, 4-N,N-diphenylamino-(2-diphenylvinyl)benzene, 3-methoxy-4′-N,N-diphenylaminostilbene, N-phenylcarbazole etc. are used.

Furthermore, 4,4′-bis[N-(1-naphtyl)-N-phenylamino]biphenyl disclosed in U.S. Pat. No. 5,061,569 and the compounds disclosed in EP-A 508,562, in which three triphenylamine units are bound to a nitrogen atom, such as 4,4′,4″-tris[N-(3-methylphenyl)-N-phenylamino]tri-phenylamine, can be used.

A positive hole transporting layer can be formed by preparing an organic film containing at least one positive hole transporting material on the anode. The positive hole transporting layer can be formed by the vacuum deposition method, the spin-coating method, the casting method, ink jet printing method, the LB method and the like. Of these methods, the vacuum deposition method, the spin-coating method and the casting method are particularly preferred in view of ease and cost.

In the case of using the vacuum deposition method, the conditions for deposition may be chosen in the same manner as described for the formation of a light emitting layer (see above). If it is desired to form a positive hole transporting layer comprising more than one positive hole transporting material, the coevaporation method can be employed using the desired compounds.

In the case of forming a positive hole transporting layer by the spin-coating method or the casting method, the layer can be formed under the conditions described for the formation of the light emitting layer (see above).

As in the case of forming the light emitting layer a smoother and more homogeneous positive hole transporting layer can be formed by using a solution containing a binder and at least one positive hole transporting material. The coating using such a solution can be performed in the same manner as described for the light emitting layer. Any polymer binder may be used, provided that it is soluble in the solvent in which the at least one positive hole transporting material is dissolved. Examples of appropriate polymer binders and of appropriate and preferred concentrations are given above when describing the formation of a light emitting layer.

The thickness of the positive hole transporting layer is preferably chosen in the range of from 0.5 to 1000 nm, preferably from 1 to 100 nm, more preferably from 2 to 50 nm.

As hole injection materials known organic hole transporting compounds such as metal-free phthalocyanine (H₂Pc), copper-phthalocyanine (Cu—Pc) and their derivatives as described, for example, in JP64-7635 can be used. Furthermore, some of the aromatic amines defined as hole transporting materials above, which have a lower ionisation potential than the hole transporting layer, can be used.

A hole injection layer can be formed by preparing an organic film containing at least one hole injection material between the anode layer and the hole transporting layer. The hole injection layer can be formed by the vacuum deposition method, the spin-coating method, the casting method, the LB method and the like. The thickness of the layer is preferably from 5 nm to 5 μm, and more preferably from 10 nm to 100 nm.

The electron transporting materials should have a high electron injection efficiency (from the cathode) and a high electron mobility. The following materials can be exemplified for electron transporting materials: tris(8-hydroxyquinolinato)-aluminum(III) and its derivatives, bis(10-hydroxybenzo[h]quinolinolato)beryllium(II) and its derivatives, oxadiazole derivatives, such as 2-(4-biphenyl)-5-(4-tert.-butylphenyl)-1,3,4-oxadiazole and its dimer systems, such as 1,3-bis(4-tert.-butylphenyl-1,3,4)oxadiazolyl)biphenylene and 1,3-bis(4-tert.-butylphenyl-1,3,4-oxadiazolyl)phenylene, dioxazole derivatives, triazole derivatives, coumarine derivatives, imidazopyridine derivatives, phenanthroline derivatives or perylene tetracarboxylic acid derivatives disclosed in Appl. Phys. Lett. 48 (2) (1986) 183.

An electron transporting layer can be formed by preparing an organic film containing at least one electron transporting material on the hole transporting layer or on the light-emitting layer. The electron transporting layer can be formed by the vacuum deposition method, the spin-coating method, the casting method, the LB method and the like.

It is preferred that the positive hole inhibiting materials for a positive hole inhibiting layer have high electron injection/transporting efficiency from the electron transporting layer to the light emission layer and also have higher ionisation potential than the light emitting layer to prevent the flowing out of positive holes from the light emitting layer to avoid a drop in luminescence efficiency.

As the positive hole inhibiting material known materials, such as Balq, TAZ and phenanthroline derivatives, e.g. bathocuproine (BCP), can be used:

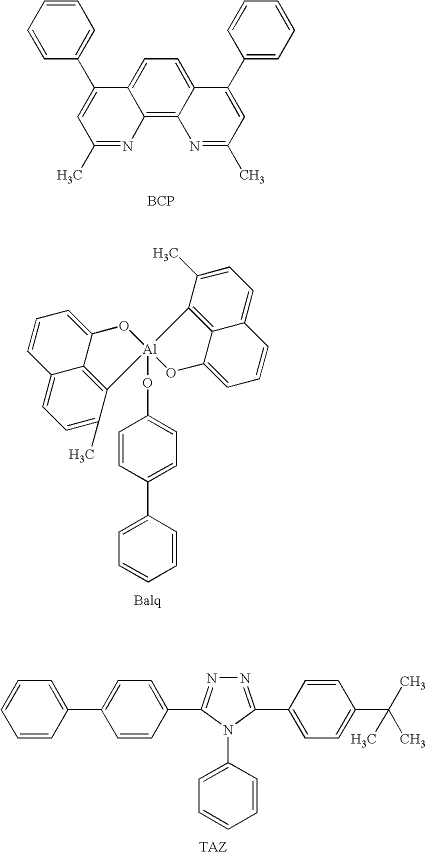

The positive hole inhibiting layer can be formed by preparing an organic film containing at least one positive hole inhibiting material between the electron transporting layer and the light-emitting layer. The positive hole inhibiting layer can be formed by the vacuum deposition method, the spin-coating method, the casting method, ink jet printing method, the LB method and the like. The thickness of the layer preferably is chosen within the range of from 5 nm to 2 μm, and more preferably, within the range of from 10 nm to 100 nm.

As in the case of forming a light emitting layer or a positive hole transporting layer, a smoother and more homogeneous electron transporting layer can be formed by using a solution containing a binder and at least one electron transporting material.

The thickness of an electron transporting layer is preferably chosen in the range of from 0.5 to 1000 nm, preferably from 1 to 100 nm, more preferably from 2 to 50 nm.

In a preferred embodiment, the host chromphore is a diketopyrrolopyrrole having a photoluminescence emission peak at 500 to 720 nm, preferably 520 to 630 nm, most preferred 540 to 600 nm. The host chromphore is preferably a diketopyrrolopyrrole of formula III.

The light-emitting compositions have a fluorescence emission maximum in the range of from 500 to 780, preferably from 520 to 750, more preferred from 540 to 700 nm. Further, the inventive compounds preferably exhibit an absorption maximum in the range of 450 to 600 nm.

The light-emitting compositions usually exhibit a fluorescence quantum yield (“FQY”) in the range of from 1>FQY≧0.3 (measured in aerated toluene or DMF). Further, in general, the inventive compositions exhibit a molar absorption coefficient in the range of from 5000 to 100000.

Another embodiment of the present invention relates to a method of coloring high molecular weight organic materials (having a molecular weight usually in the range of from 10³ to 10⁷ g/mol; comprising biopolymers, and plastic materials, including fibres) by incorporating therein the inventive compounds or compositions by methods known in the art.

The inventive compounds and compositions can be used, as described, for example, for DPP compounds in EP-A-1087005, for the preparation of
inks, for printing inks in printing processes, for flexographic printing, screen printing, packaging printing, security ink printing, intaglio printing or offset printing, for pre-press stages and for textile printing, for office, home applications or graphics applications, such as for paper goods, for example, for ballpoint pens, felt tips, fiber tips, card, wood, (wood) stains, metal, inking pads or inks for impact printing processes (with impact-pressure ink ribbons), for the preparation of
colorants, for coating materials, for industrial or commercial use, for textile decoration and industrial marking, for roller coatings or powder coatings or for automotive finishes, for high-solids (low-solvent), water-containing or metallic coating materials or for pigmented formulations for aqueous paints, for the preparation of
pigmented plastics for coatings, fibers, platters or mold carriers, for the preparation of non-impact-printing material for digital printing, for the thermal wax transfer printing process, the ink jet printing process or for the thermal transfer printing process, and also for the preparation of
color filters, especially for visible light in the range from 400 to 700 nm, for liquid-crystal displays (LCDs) or charge combined devices (CCDs) or for the preparation of cosmetics or for the preparation of
polymeric ink particles, toners, dye lasers, dry copy toners liquid copy toners, or electrophotographic toners, and electroluminescent devices.

Another preferred embodiment concerns the use of the inventive compounds and compositions for color changing media. There are three major techniques in order to realize full-color organic electroluminescent devices:

(i) use of the three primary colors blue, green and red generated by electroluminescence,
(ii) conversion of the electroluminescent blue or white to photoluminescent green and red via color changing media (CCM), which absorb the above electroluminescent blue, and fluorescence in green and red.
(iii) conversion of the white luminescent emission to blue, green and red via color filters.

The inventive compounds or compositions are useful for EL materials for the above category (i) and, in addition, for the above mention technique (ii). This is because the invented compounds or compositions can exhibit strong photoluminescence as well as electroluminescence.

Technique (ii) is, for example, known from U.S. Pat. No. 5,126,214, wherein EL blue with a maximum wavelength of ca. 470-480 nm is converted to green and red using coumarin, 4 (dicyanomethylene)-2-methyl-4-(p-dimethylaminostyryl)-4H-pyran, pyridine, rhodamine 6G, phenoxazone or other dyes.

The inventive compounds or compositions are useful for EL materials for the above category (iii) as an element of white luminescent in combination of other compensatory electroluminescence to construct white luminescent. This is because compounds or compositions can exhibit strong photoluminescence as well as electroluminescence.

Illustrative examples of suitable organic materials of high molecular weight which can be colored with the inventive compositions are described in EP-A-1087005.

Particularly preferred high molecular weight organic materials, in particular for the preparation of a paint system, a printing ink or ink, are, for example, cellulose ethers and esters, e.g. ethylcellulose, nitrocellulose, cellulose acetate and cellulose butyrate, natural resins or synthetic resins (polymerization or condensation resins) such as aminoplasts, in particular urea/formaldehyde and melamine/formaldehyde resins, alkyd resins, phenolic plastics, polycarbonates, polyolefins, polystyrene, polyvinyl chloride, polyamides, poly-urethanes, polyester, ABS, ASA, polyphenylene oxides, vulcanized rubber, casein, silicone and silicone resins as well as their possible mixtures with one another.

It is also possible to use high molecular weight organic materials in dissolved form as film formers, for example boiled linseed oil, nitrocellulose, alkyd resins, phenolic resins, melamine/formaldehyde and urea/formaldehyde resins as well as acrylic resins.

Said high molecular weight organic materials may be obtained singly or in admixture, for example in the form of granules, plastic materials, melts or in the form of solutions, in particular for the preparation of spinning solutions, paint systems, coating materials, inks or printing inks.

In a particularly preferred embodiment of this invention, the inventive compounds and compositions are used for the mass coloration of polyvinyl chloride, polyamides and, especially, polyolefins such as polyethylene and polypropylene as well as for the preparation of paint systems, including powder coatings, inks, printing inks, color filters and coating colors.

Illustrative examples of preferred binders for paint systems are alkyd/melamine resin paints, acryl/melamine resin paints, cellulose acetate/cellulose butyrate paints and two-pack system lacquers based on acrylic resins which are crosslinkable with polyisocyanate.

Hence, another embodiment of the present invention relates to a composition comprising
(a) 0.01 to 50, preferably 0.01 to 5, particularly preferred 0.01 to 2% by weight, based on the total weight of the coloured high molecular organic material, of a compound according to formula I or of a composition according to the present invention, and
(b) 99.99 to 50, preferably 99.99 to 95, particularly preferred 99.99 to 98% by weight, based on the total weight of the coloured high molecular organic material, of a high molecular organic material, and
(c) optionally, customary additives such as rheology improvers, dispersants, fillers, paint auxiliaries, siccatives, plasticizers, UV-stabilizers, and/or additional pigments or corresponding precursors In effective amounts, such as e.g. from 0 to 50% by weight, based on the total weight of (a) and (b).

To obtain different shades, the inventive (fluorescent compounds) of formula I or the inventive compositions may advantageously be used in admixture with fillers, transparent and opaque white, colored and/or black pigments as well as customary luster pigments in the desired amount.

For the preparation of the paint systems, coating materials, color filters, inks and printing inks, the corresponding high molecular weight organic materials, such as binders, synthetic resin dispersions etc. and the inventive compounds or compositions are usually dispersed or dissolved together, if desired together with customary additives such as dispersants, fillers, paint auxiliaries, siccatives, plasticizers and/or additional pigments or pigment precursors, in a common solvent or mixture of solvents. This can be achieved by dispersing or dissolving the individual components by themselves, or also several components together, and only then bringing all components together, or by adding everything together at once.

Hence, a further embodiment of the present invention relates to a method of using the inventive compounds or compositions for the preparation of dispersions and the corresponding dispersions, and paint systems, coating materials, color filters, inks and printing inks comprising the inventive compositions.

A particularly preferred embodiment relates to the use of the inventive compounds, or compositions for the preparation of fluorescent tracers for e.g. leak detection of fluids such as lubricants, cooling systems etc., as well as to fluorescent tracers or lubricants comprising the inventive compositions.

For the pigmentation of high molecular weight organic material, the inventive compounds or compositions, optionally in the form of masterbatches, are mixed with the high molecular weight organic materials using roll mills, mixing apparatus or grinding apparatus. Generally, the pigmented material is subsequently brought into the desired final form by conventional processes, such as calandering, compression molding, extrusion, spreading, casting or injection molding.

For pigmenting lacquers, coating materials and printing inks the high molecular weight organic materials and the inventive compounds or compositions, alone or together with additives, such as fillers, other pigments, siccatives or plasticizers, are generally dissolved or dispersed in a common organic solvent or solvent mixture. In this case it is possible to adopt a procedure whereby the individual components are dispersed or dissolved individually or else two or more are dispersed or dissolved together and only then are all of the components combined.

The present invention additionally relates to inks comprising a coloristically effective amount of the pigment dispersion of the inventive compositions.

The weight ratio of the pigment dispersion to the ink in general is chosen in the range of from 0.001 to 75% by weight, preferably from 0.01 to 50% by weight, based on the overall weight of the ink.

The preparation and use of color filters or color-pigmented high molecular weight organic materials are well-known in the art and described e.g. in Displays 14/2, 1151 (1993), EP-A 784085, or GB-A 2,310,072.

The color filters can be coated for example using inks, especially printing inks, which can comprise pigment dispersions comprising the inventive compositions or can be prepared, for example, by mixing a pigment dispersion comprising an inventive composition with chemically, thermally or photolytically structurable high molecular weight organic material (so-called resist). The subsequent preparation can be carried out, for example, in analogy to EP-A 654 711 by application to a substrate, such as a LCD (liquid crystal display), subsequent photostructuring and development.

Particular preference for the production of color filters is given to pigment dispersions comprising an inventive compound or composition which possess non-aqueous solvents or dispersion media for polymers.

The present invention relates, moreover, to toners comprising a pigment dispersion containing an inventive compounder composition or a high molecular weight organic material pigmented with an inventive composition in a coloristically effective amount.

The present invention additionally relates to colorants, colored plastics, polymeric ink particles, or non-impact-printing material comprising an inventive composition, preferably in the form of a dispersion, or a high molecular weight organic material pigmented with an inventive composition in a coloristically effective amount.

A coloristically effective amount of the pigment dispersion according to this invention comprising an inventive composition denotes in general from 0.0001 to 99.99% by weight, preferably from 0.001 to 50% by weight and, with particular preference, from 0.01 to 50% by weight, based on the overall weight of the material pigmented therewith.

The inventive compositions can be applied to colour polyamides, because they do not decompose during the incorporation into the polyamides. Further, they exhibit an exceptionally good lightfastness, a superior heat stability, especially in plastics.

The organic EL device of the present invention has significant industrial values since it can be adapted for a flat panel display of an on-wall television set, a flat light-emitting device, a light source for a copying machine or a printer, a light source for a liquid crystal display or counter, a display signboard and a signal light. The compounds and compositions of the present invention can be used in the fields of an organic EL device, an electrophotographic photoreceptor, a photoelectric converter, a solar cell, an image sensor, and the like.

The following examples are for illustrative purposes only and are not to be construed to limit the scope of the instant invention in any manner whatsoever. In the examples the “parts” denote “parts by weight” and the “percentages” denote “percentages by weight”, unless otherwise stated.

### EXAMPLE 1

5.0 g (13 mmol) of 2,9-dichloroquinacridone (Pigment Red 202), 1.53 g (39 mmol) of NaH (60% assay) and 100 ml of dry N-methylpyrrolidone (NMP) are placed in a three necked flask and stirred vigorously by a mechanical stirrer at 80° C. under nitrogen for 15 hours. The reaction mixture is allowed to cool to ambient temperature, 12 g (60 mmol) of 1-iodohexane are added and the reaction mixture is stirred for 5 hours. After the reaction has been completed, 100 ml of water are added. The reaction mixture is filtered and washed with methanol until the extracts become colorless. After drying the crude product is purified by column chromatography, whereby 680 mg of QA-1 are obtained as red powder.

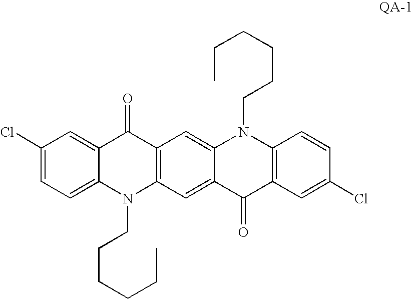

0.33 g (0.6 mmol) of QA-1, 160 mg (1.68 mmol) sodium tert-butoxide and 0.53 g (3.12 mmol) diphenylamine are added to 10 ml o-xylene. The suspension is stirred and degassed using vacuum. To this are added 14 mg (0.06 mmol) [(allyl)PdBr(P(iPr)₃)]. The suspension is heated to 120° C. and stirred for two hours at this temperature. TLC showed complete conversion. The purple mixture is cooled to ambient temperature, poured into water and diluted with CH₂Cl₂. The aqueous phase is extracted twice with CH₂Cl₁. The combined organic phases are dried with Na₂SO₄, filtered and evaporated to dryness. After purification by column chromatography (hexane/CH₂Cl₂/MeOH 5:1:0.2) 0.32 g (65%) of a purple solid are obtained. ¹H-NMR and MS showed the desired compound to be pure.

¹H-NMR (in CDCl₃): δ 8.7 (s, 2H), 8.3 (d, 2H), 7.6 (dd, 2H), 7.5 (d, 2H), 7.3 (m, 8H), 7.1 (d, 8H), 7.0 (t, 4H), 4.5 (t, 4H), 2.0 (m, 4H), 1.6 (m, 4H), 1.4 (m, 8H), 0.9 (t. 6H).

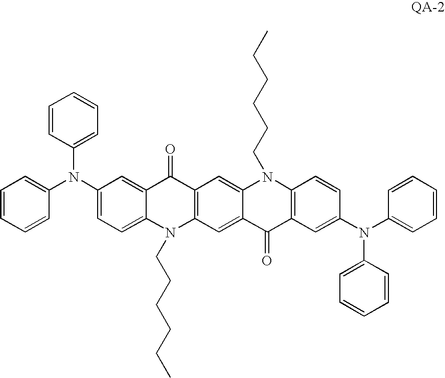

### EXAMPLE 2

0.33 g (0.6 mmol) of QA-1, 160 mg (1.68 mmol) sodium tert-butoxide and 1.2 g (3.12 mmol) 4,4′-bis(α,α-dimethylbenzyl) diphenylamine are added to 10 ml o-xylene. The suspension is stirred and degassed using vacuum. To this is added 7 mg (0.03 mmol) [(allyl)PdBr(P(iPr)₃)]. The suspension is heated to 120° C. and stirred for one hour at this temperature. TLC showed ca. 30% starting material left A second portion of 7 mg catalyst is added and the reaction mixture is heated for an additional hour at 120° C. after which TLC showed complete conversion. The purple mixture was cooled to ambient temperature, poured into water, diluted with CH₂Cl₂, and filtered over cotton. The aqueous phase was extracted twice with CH₂Cl₂. The combined organic phases are dried with Na₂SO₄, filtered and evaporated to dryness. After purification by column chromatography (hexane/CH₂Cl₂/MeOH 5:1:0.2) 0.24 g (31%) of a purple solid are obtained. ¹H-NMR and MS showed the desired compound to be pure.

¹H-NMR (in CDCl₃): 38.7 (s, 2H), 8.3 (d, 2H), 7.6 (dd, 2H), 7.4 (d, 2H), 7.3 (d, 16H), 7.2 (m, 4H), 7.1 (m, 8H), 7.0 (m, 8H) 4.5 (t, 4H), 2.0 (m, 4H), 1.7 (s, 24H), 1.6 (m, 4H) 1.5 (m, 8H), 0.9 (t, 6H).

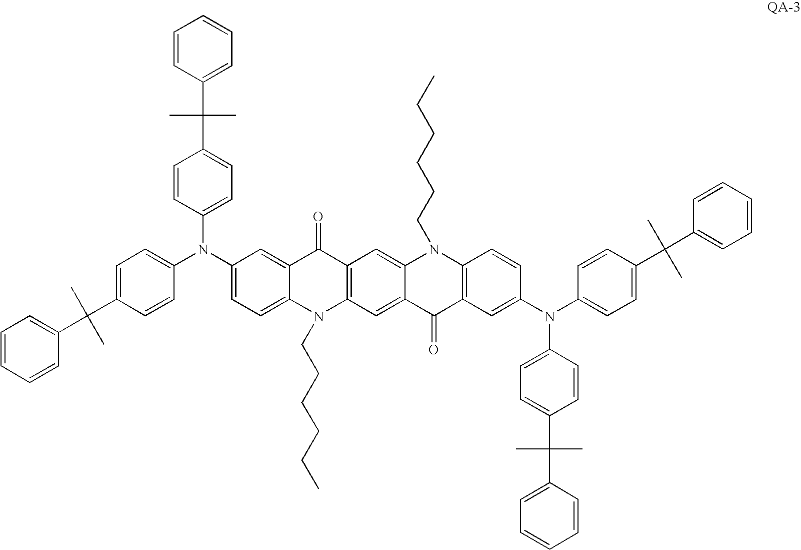

### EXAMPLE 3

0.33 g (0.6 mmol) of QA-1, 160 mg (1.68 mmol) sodium tert-butoxide and 0.62 g (3.12 mmol) 4,4′-dimethyl diphenylamine are added to 10 ml o-xylene. The suspension is stirred and degassed using vacuum. To this is added 14 mg (0.06 mmol) [(allyl)PdBr(P(iPr)₃)]. The suspension is heated to 120° C. and stirred for two hours at this temperature. TLC showed complete conversion. The purple mixture is cooled to ambient temperature, poured into water and diluted with CH₂Cl₂. The aqueous phase is extracted twice with CH₂Cl₂. The combined organic phases are dried with Na₂SO₄, filtered and evaporated to dryness. After purification by column chromatography (hexane/CH₂Cl₂/MeOH 5:1:0.2) 0.35 g (67%) of a purple solid are obtained. ¹H-NMR and MS showed the desired compound to be pure. ¹H-NMR (in CDCl₃): a 8.7 (s, 2H), 8.2 (d, 2H), 7.5 (dd, 2H), 7.4 (d, 2H), 7.0 (m, 16H), 4.5 (t, 4H), 2.3 (s, 12H), 2.0 (m, 4H) 1.6 (m, 4H), 1.4 (m, 8H), 0.9 (t, 6H).

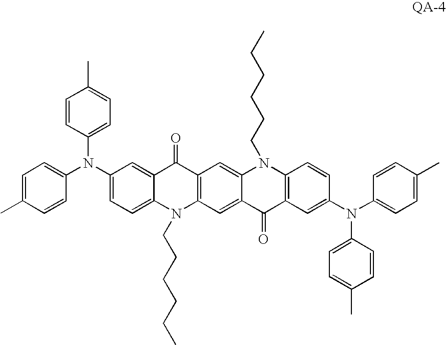

### EXAMPLE 4

0.34 g (0.62 mmol) OA-1, 170 mg (1.73 mmol) sodium tert-butoxide and 0.54 g (2.47 mmol) N-phenyl naphthyl amine are added to 10 ml o-xylene. The suspension is stirred and degassed using vacuum. To this are added 14 mg (0.06 mMol) [(allyl)PdBr(P(iPr)₃)]. The suspension is heated to 120° C. and stirred for two hours at this temperature. TLC showed complete conversion. The purple mixture is cooled to ambient temperature, poured into water and diluted with CH₂Cl₂. The aqueous phase is extracted twice with CH₂Cl₂. The combined organic phases are dried with Na₂SO₄, filtered and evaporated to dryness. After purification by column chromatography (hexane/CH₂Cl₂/MeOH 5:1:0.2) 0.30 g (53%) of a purple solid are obtained. ¹H-NMR and MS showed the desired compound to be pure.

¹H-NMR (in CDCl₃): δ=8.7 (s, 2H), 8.3 (d, 2H), 7.8 (t, 4H), 7.6 (m, 4H), 7.5-7.2 (m, 14H), 7.1 (d, 4H), 7.0 (t, 2H), 4.5 (t, 4H), 2.0 (m, 4H), 1.6 (m, 4H), 1.4 (m, 8H), 0.9 (t, 6H).

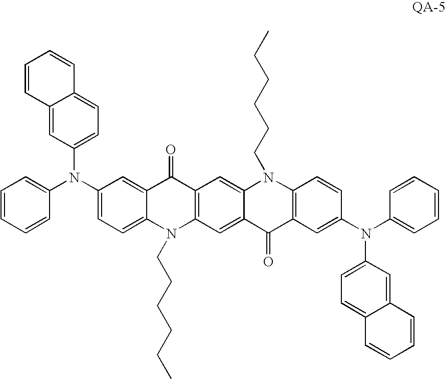

### EXAMPLE 5

a) 5.0 g (13 mmol) of Pigment Red 202, 1.56 g (39 mmol) of NaH (60% assay) and 50 ml of dry NMP are placed in a three necked flask and stirred vigorously by a mechanical stirrer at 120° C. under nitrogen for 15 hours. After the reaction mixture has been allowed to cool to ambient temperature 5.5 g (39 mmol) of methyl iodide are added and stirred for 5 hours. After the reaction has been completed, 100 ml of water are added and filtered and washed with methanol until the extracts become colorless. After drying the crude product is purified via sublimation and 2.7 g of QA-7 are obtained as a red brilliant powder,

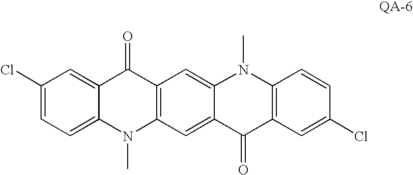

b) 0.49 g (1.2 mmol) of QA-6, 160 mg (1.68 mmol) sodium tert-butoxide and 0.53 g (3.12 mmol) diphenylamine are added to 10 ml o-xylene. The suspension is stirred and degassed using vacuum. To this is added 18 mg (0.076 mmol) [(allyl)PdBr(P(iPr)₃)]. The suspension is heated to 120° C. and stirred for two hours at this temperature. TLC showed complete conversion. The purple mixture is cooled to ambient temperature, poured into water and diluted with CH₂Cl₂. The aqueous phase is extracted twice with CH₂Cl₂. The combined organic phases are dried with Na₂SO₄, filtered and evaporated to dryness. After purification by column chromatography (hexane/CH₂Cl₂/MeOH 5:1:0.2) 0.07 g (9%) of a purple solid are obtained. ¹H-NMR and MS showed the desired compound to be pure. ¹H-NMR (in CDCl₃): δ 8.7 (s, 2H), 8.3 (d, 2H), 7.6 (dd, 2H), 7.5 (d, 2H), 7.3 (m, 8H), 7.1 (d, 8H), 7.0 (t, 4H), 4.0 (s, 6H).

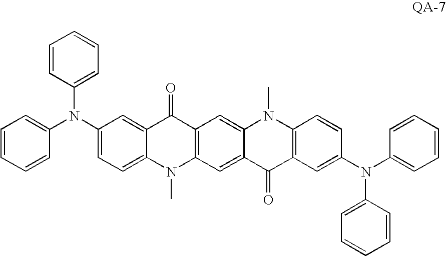

### EXAMPLE 6

A glass substrate (manufactured by Geomatek Co., a product prepared by electron beam vapor deposition method) on which an ITO transparent electroconductive film had been deposited up to a thickness of ca. 150 nm is cut into a size of 10×20 mm, and etched. The substrate thus obtained is subjected to ultrasonic washing with detergent water for 15 minutes, and then washing with pure water. Subsequently, the substrate is subjected to ultrasonic washing with acetone for 15 minutes, and then dried. Just before forming the substrate into an element, the substrate thus obtained is subjected to a plasma treatment for half an hour and placed in a vacuum vapour deposition apparatus, and the apparatus is evacuated until the inner pressure reached 1×10⁻⁵ Pa or less. Then, according to the resistance heating method, firstly CuPc (20 nm) and N,N′-diphenyl-N,N′-(1-naphtyl)-1,1′-diphenyl-44′-diamine (α-NPD) are vapor-deposited successively as a positive hole transporting material up to a thickness of 40 nm, to form a positive hole transporting layer. Subsequently, H-2, a DPP compound of formula III, and QA-2 are co-deposited as a light emitting layer up to a thickness of 30 nm by controlling the ratio of deposition rate (H-2: QA-2=99: ca. 1) to form an uniform light emitting layer. Subsequently, a Alq₃ layer is vapor-deposited to form an electron transporting/injection layer having a thickness of 30 nm. In addition, LiF was deposited on Alq3 layer with a thickness of 0.5 nm. On top of that, a Mg—Ag alloy (10:1) is vapor-deposited to form a cathode having a thickness of 150 nm, whereby an element having a size of 5×5 mm square is prepared. The luminescent peak wavelength and emission intensity of the luminescent element thus obtained is summarized in Table 1.

### EXAMPLE 7

Example 6 is repeated, except that the emitting material of example 6 is replaced by the emitting materials as described in table 1.

TABLE 1

| | Emitting Material | | | |
|---|---|---|---|---|
| Device of | Compound | Compound | EL properties | |
| Example | [99 wt %] | [ca. 1 wt %] | Peak (nm) | Intensity (cd/m²) |
| Ex. 7 | H-1 | QA-2 | 615 | 11340 |
| Ex. 8 | H-1 | QA-5 | 615 | 8681 |

### REFERENCE EXAMPLE 1

Example 8 is repeated, except that the compound below (A-3; Example 81 of EP-A-1087006) is used as the light emitting material. The maximum luminance is 5260 Cd/m².

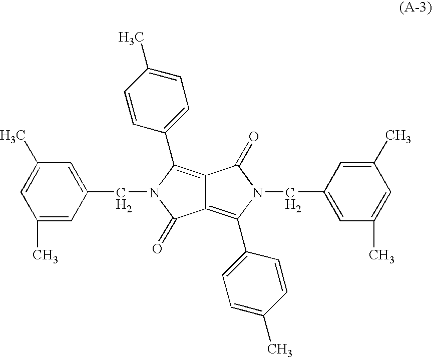

1. A compound of formula 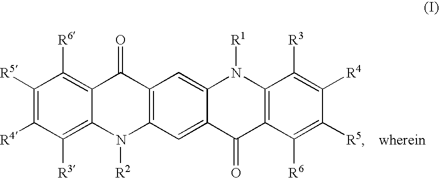
R¹ and R² may be the same or different and are selected from a C₁-C₂₅alkyl group, which can be substituted by fluorine, chlorine or bromine, an allyl group, which can be substituted one to three times with C₁-C₄alkyl, a cycloalkyl group, a cycloalkyl group, which can be condensed one or two times by phenyl which can be substituted one to three times with C₁-C₄-alkyl, halogen, nitro or cyano, an alkenyl group, a cycloalkenyl group, an alkynyl group, a haloalkyl group, a haloalkenyl group, a haloalkynyl group, a ketone or aldehyde group, an ester group, a carbamoyl group, a ketone group, a silyl group, a siloxanyl group, A³ or —CR⁷R⁸—(CH₂)ₘ-A³, wherein
R⁷ and R⁸ independently from each other stand for hydrogen, or C₁-C₄alkyl, or phenyl, which can be substituted one to three times with C₁-C₄alkyl,
A³ stands for aryl or heteroaryl which can be substituted one to three times with C₁-C₈alkyl and/or C₁-C₈alkoxy, and m stands for 0, 1, 2, 3 or 4,
R³, R^{3′}, R⁶ and R^{6′}, independently of one another, represent hydrogen, halogen, C₁-C₁₈alkyl, halogen-substituted C₁-C₁₈alkyl, C₁-C₁₈alkoxy, C₁-C₁₈alkylthio, cycloalkyl, optionally substituted aryl or arylalkyl, wherein the substituents are alkoxy, halogen or alkyl,
R⁴ and R^{4′} are independently of each other R³, or a group —NAr¹Ar²,
R⁵ and R^{5′} are independently of each other R³, or a group —NAr³Ar⁴, or 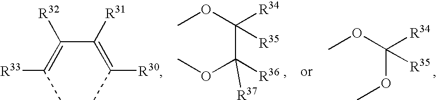
R^{3′} and R^{4′} and/or R³ and R⁴ together are a group or 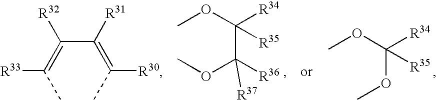
R^{5′} and R^{6′} and/or R⁵ and R⁶ together are a group wherein
R³⁰, R³¹, R³² and R³³ are independently of each other hydrogen, C₁-C₁₈alkyl, halogen-substituted C₁-C₁₈alkyl, C₁-C₁₈alkoxy, or C₁-C₂₈alkylthio,
R³⁴, R³⁵, R³⁶ and R³⁷ are independently of each other hydrogen, C₁-C₁₈alkyl, halogen-substituted C₁-C₁₈alkyl, C₁-C₁₈alkoxy, or C₁-C₂₈alkylthio,
Ar¹, Ar², Ar³ and Ar⁴ are independently of each other an aryl group, which can optionally be substituted, or a heteroaryl group, which can optionally be substituted, with the proviso that at least one of the groups R⁴, R^{4′}, R⁵ and R^{5′} is a group —NAr¹Ar², or —NAr³Ar⁴, and compounds of formula 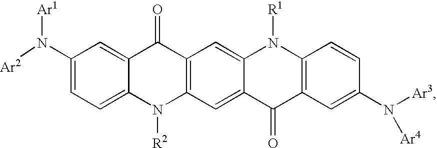
wherein R¹═R²═C₂H₅ and 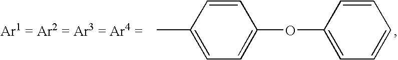
and R¹═R²=Ph and Ar¹=Ar²=Ar³=Ar⁴═ 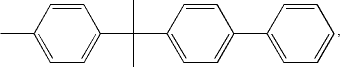
are excluded.

2. A compound according to claim 1 of formula 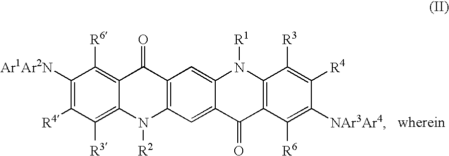
Ar¹, Ar², Ar³ and Ar⁴ are independently of each other a group —Ar⁵—X¹—Ar⁶, 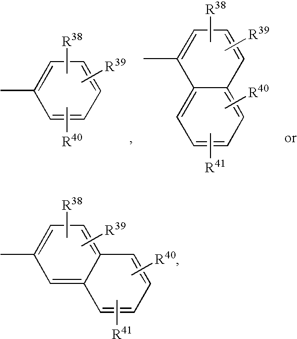
or Ar¹ and Ar² and/or Ar³ and Ar⁴ together with the nitrogen atom to which they are bonded form a five or six membered heterocyclic ring
which can be condensed by one or two optionally substituted phenyl groups, wherein
X¹ is —C(X²)(X³)—, —O—, —S—, —SO₂—, —C(═O)— 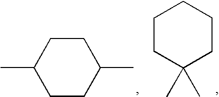
—(C₂H₂ₓ)—O—(C_{y}H_{2y})—, wherein each of x and y is an integer of 0 to 20, while x+y=0 in no case, a substituted or unsubstituted alkylene group having at least two carbon atoms, a substituted or unsubstituted alkylidene group having at least two carbon atoms, a substituted or unsubstituted alicyclic group having at least two carbon atoms, 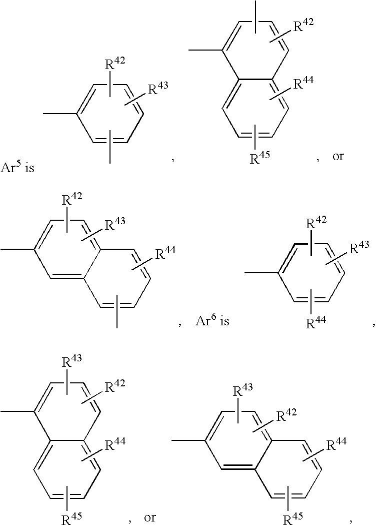
X² and X³ independently from each other stand for hydrogen, C₁-C₁₈alkyl, halogen-substituted C₁-C₁₈alkyl, or phenyl, which can be substituted one to three times with C₁-C₈alkyl and/or C₁-C₈alkoxy,
R³⁸, R³⁹, R⁴⁰, R⁴, R⁴², R⁴³, R⁴⁴ and R⁴⁵ independently from each other stands for hydrogen, C₁-C₈-alkyl, C₁-C₈-alkoxy, or phenyl.

3. A compound according to claim 2, wherein Ar¹, Ar², Ar³ and Ar⁴ are independently of each other a group 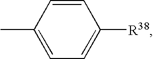
wherein R³⁸ is hydrogen, or C₁-C₄alkyl;

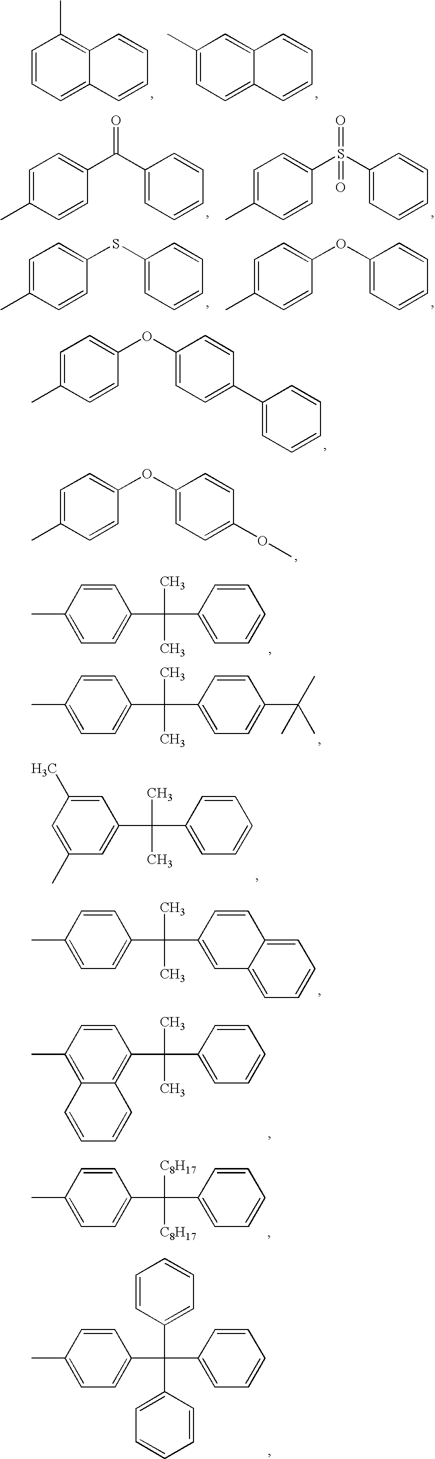

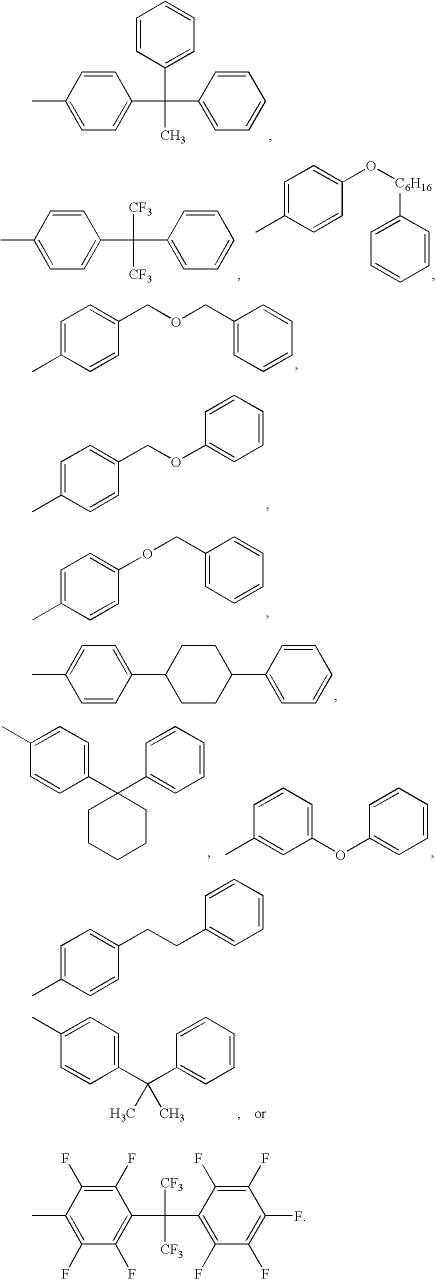

4. A compound according to claim 1, wherein R¹ and R² may be the same or different and are selected from a C₁-C₁₈alkyl group a C₅-C₁₂cyoloalkyl group which can be substituted one to three times with C₁-C₄-alkyl, or C₁-C₄-alkoxy, or a cycloalkyl group which can be condensed one or two times by phenyl which can be substituted one to three times with C₁-C₈-alkyl, C₁-C₈-alkoxy, halogen and cyano, a C₅-C₁₂cycloalkenyl group, which can be substituted one to three times with C₁-C₄-alkyl, or C₁-C₄-alkoxy; a C₆-C₁₄aryl group which can be substituted one to three times by C₁-C₈alkyl, or C₁-C₈alkoxy; or —CR⁷R⁸—(CH₂)ₘ-A³ wherein
R⁷ and R⁸ stand for hydrogen, or C₁-C₄alkyl, A³ stands for phenyl, 1- or 2-naphthyl, which can be substituted one to three times by C₁-C₈alkyl, or C₁-C₈alkoxy; and m stands for 0, or 1.

5. A compound according to claim 1, which is selected from

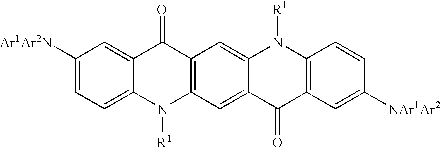

| Compound | R¹ | Ar¹ | Ar² |
|---|---|---|---|
| QA-2 | n-C₆H₁₃ | phenyl | phenyl |
| QA-3 | n-C₆H₁₃ | 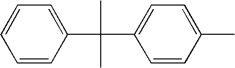 | 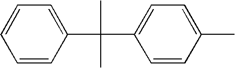 |
| QA-4 | n-C₆H₁₃ | tolyl | tolyl |
| QA-5 | n-C₆H₁₃ | 2-naphthyl | phenyl |
| QA-7 | CH₃ | phenyl | phenyl |
| QA-10 | CH₃ | 2-naphthyl | phenyl |
| QA-11 | C₂H₅ | phenyl | phenyl |
| QA-12 | phenyl | phenyl | phenyl |
| QA-13 | 2-cyclohexene | phenyl | phenyl |
| QA-14 | 2-cyclohexene | 1-naphthyl | phenyl |
| QA-15 | 2-cyclohexene | 4-tolyl | 4-tolyl |
| QA-16 | 2-cyclohexene | 2-naphthyl | phenyl |
| QA-17 | cyclohexane | phenyl | phenyl |
| QA-18 | cyclohexane | 1-naphthyl | phenyl |
| QA-19 | cyclohexane | 4-tolyl | 4-tolyl |
| QA-20 | cyclohexane | 2-naphthyl | phenyl |

6. A composition comprising a guest chromophore and a host chromophore, wherein the absorption spectrum of the guest chromophore overlaps with the fluorescence emission spectrum of the host chromophore, wherein the host chromophore is a diketopyrrolopyrrole having a photoluminescence emission peak at 500 to 720 nm, and wherein the guest chromophore is a compound of formula I according to claim 1.

7. A composition according to claim 6, wherein the host chromophore is a diketopyrrolopyrrole (“DPP”) represented by formula 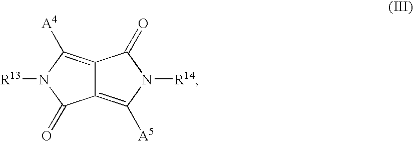
wherein R¹³ and R¹⁴ independently from each other stand for C₁-C₂₅-alkyl, which can be substituted by fluorine, chlorine or bromine, C₅-C₁₂-cycloalkyl or C₅-C₁₂-cycloalkyl, which can be condensed one or two times by phenyl which can be substituted one to three times with C₁-C₄-alkyl, halogen, nitro or cyano, silyl, A⁶ or —CR¹¹R¹²—(CH₂)ₘ-A⁶, wherein R¹¹ and R¹² independently from each other stand for hydrogen, fluorine, chlorine, bromine, cyano or C₁-C₄alkyl, which can be substituted by fluorine, chlorine or bromine, or phenyl which can be substituted one to three times with C₁-C₄alkyl, A⁶ stands for phenyl or 1- or 2-naphthyl which can be substituted one to three times with C₁-C₈alkyl, C₁-C₈alkoxy, halogen, nitro, cyano, phenyl, which can be substituted with C₁-C₈alkyl or C₁-C₈alkoxy one to three times, —NR²³R²⁴, wherein R²³ and R²⁴ represent hydrogen, C₁-C₂₅-alkyl, C₅-C₁₂-cycloalkyl or phenyl or 1- or 2-naphthyl which can be substituted one to three times with C₁-C₈alkyl, C₁-C₈alkoxy, halogen or cyano, or phenyl, which can be substituted with C₁-C₈alkyl or C₁-C₈alkoxy one to three times, and m stands for 0, 1, 2, 3 or 4,
A⁴ and A⁵ independently from each other stand for 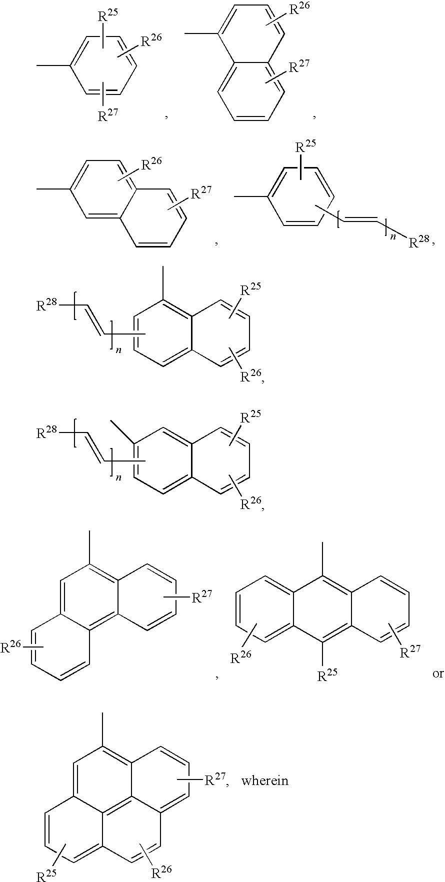
R²⁵, R²⁶, R²⁷ independently from each other stands for hydrogen, C₁-C₂₅alkyl, —CR¹¹R¹²—(CH₂)ₘ-A⁶, cyano, halogen, —OR⁹, —S(O)ₚR³⁰, or phenyl, which can be substituted one to three times with C₁-C₈alkyl or C₁-C₈alkoxy, wherein R²⁹ stands for C₁-C₂₅-alkyl, C₅-C₁₂-cycloalkyl, —CR¹¹R¹²-(CH₂)ₘ—Ph, C₆-C₂₄-aryl, or a saturated or unsaturated heterocyclic radical comprising five to seven ring atoms, wherein the ring consists of carbon atoms and one to three hetero atoms selected from the group consisting of nitrogen, oxygen and sulfur, R³⁰ stands for C₁-C₂₅-alkyl, C₅-C₁₂-cycloalkyl, —CR¹¹R¹²—(CH₂)ₘ—Ph, R²⁸ stands for C₂-C₂₀-heteroaryl or C₆-C₂₄-aryl, p stands for 0, 1, 2 or 3, m and n stands for 0, 1, 2, 3 or 4.

8. A composition according to claim 7, wherein R¹³ and R¹⁴ independently from each other stand for C₁-C₈alkyl, C₅-C₁₂-cycloalkyl, which can be substituted one to three times with C₁-C₈alkyl and/or C₁-C₈alkoxy, phenyl or 1- or 2-naphthyl which can be substituted one to three times with C₁-C₈alkyl and/or C₁-C₈alkoxy, or —CR¹¹R¹²—(CH₂)ₘ-A⁶ wherein R¹¹ and R¹² stand for hydrogen, or C₁-C₄alkyl, A⁶ stands for phenyl or 1- or 2-naphthyl, which can be substituted one to three times with C₁-C₈alkyl and/or C₁-C₈alkoxy, and m stands for 0 or 1.

9. A composition according to claim 6, wherein A⁴ and A⁵ independently from each other stand for 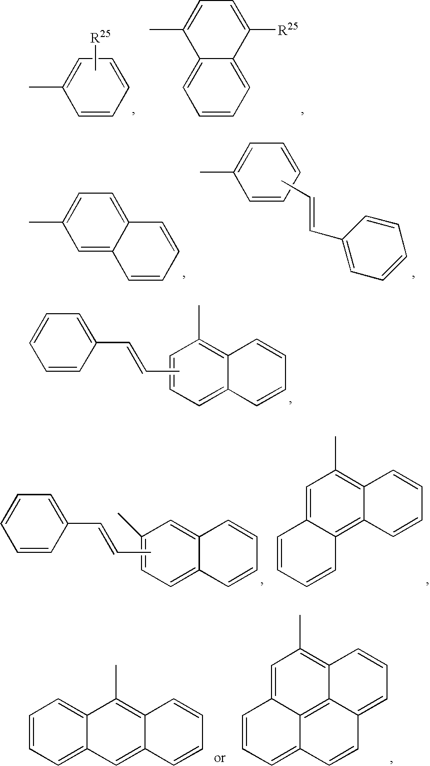
wherein R²⁵ is C₁-C₈-alkyl, phenyl, 1- or 2-naphthyl.

10. A composition according to claim 6, wherein the host chromophore is a diketopyrrolopyrrole represented by formula 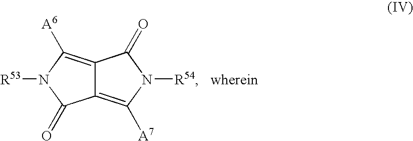
R⁵³ and R⁵⁴ may be the same or different and are selected from a C₁-C₂₅alkyl group, which can be substituted by fluorine, chlorine or bromine, an allyl group, which can be substituted one to three times with C₁-C₄alkyl, a cycloalkyl group, or a cycloalkyl group, which can be condensed one or two times by phenyl which can be substituted one to three times with C₁-C₄-alkyl, halogen, nitro or cyano, an alkenyl group, a cycloalkenyl group, an alkynyl group, a haloalkyl group, a haloalkenyl group, a haloalkynyl group, a ketone or aldehyde group, an ester group, a carbamoyl group, a ketone group, a silyl group, a siloxanyl group, A⁸ or —CR⁶⁰R⁶¹—(CH₂)ₘ-A⁸, wherein
R⁶⁰ and R⁶¹ independently from each other stand for hydrogen or C₁-C₄alkyl, or phenyl, which can be substituted one to three times with C₁-C₄alkyl,
A⁸ stands for aryl or heteroaryl, in particular phenyl or 1- or 2-naphthyl which can be substituted one to three times with C₁-C₈alkyl and/or C₁-C₈alkoxy, and m stands for 0, 1, 2, 3 or 4,
A⁶ and A⁷ independently from each other are selected from 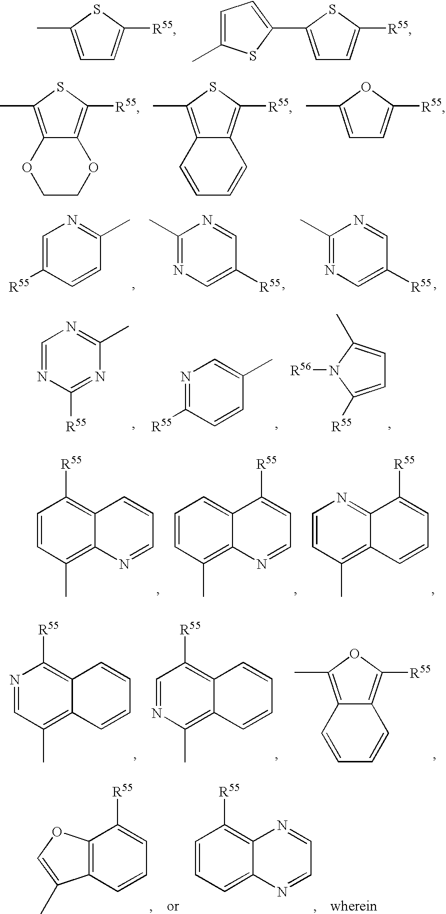
R⁵⁵ is a hydrogen atom, a C₁-C₁₂alkyl group, a C₁-C₈alkoxy group, a group of formula 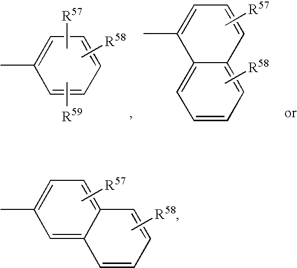
wherein R⁵⁷, R⁵⁸ and R⁵⁹ independently from each other stands for hydrogen, C₁-C₈-alkyl, or C₁-C₈-alkoxy, and R⁵⁶ stands for hydrogen, or C₁-C₈-alkyl.

11. An EL device comprising a compound according to claim 1.

12. Composition comprising
(a) 0.01 to 50% weight, based on the total weight of the colored high molecular weight organic material, of a compound according to claim 1,
(b) 99.99 to 50% by weight, based on the total weight of the colored high molecular weight organic material, of a high molecular weight organic material, and
(c) optionally customary additives in effective amounts.

13. A fluorescent tracer, color changing medium, solid dye laser or EL laser comprising a fluorescent diketopyrrolopyrrole according to claim 1.

14. A compound according to claim 2, wherein when Ar¹ and Ar² and/or Ar³ and Ar⁴ together with the nitrogen atom to which they are bonded form a five or six membered heterocyclic ring, the
ring is of the formula 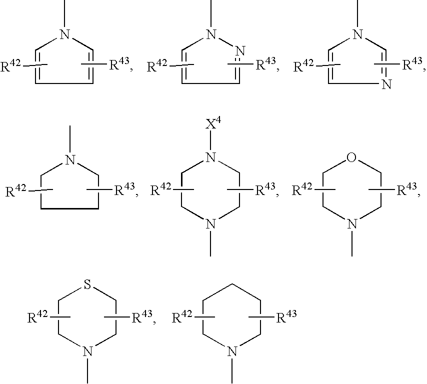
or is a heterocycic ring condensed by two optionally substituted phenyl groups of the formula 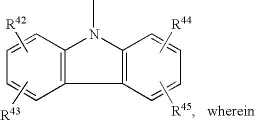
X⁴ stands for C₁-C₁₈alkyl, halogen-substituted C₁-C₁₈alkyl, or phenyl, which can be substituted one to three times with C₁-C₈alkyl and/or C₁-C₈alkoxy.

15. A compound according to claim 4, wherein R¹ and R² are independently selected from methyl, ethyl, n-propyl, isopropyl, n-butyl, sec.-butyl, isobutyl, tert.-butyl, n-pentyl, 2-pentyl, 3-pentyl, 2,2-dimethylpropyl, n-hexyl, n-heptyl, n-octyl, 1,1,3,3-tetramethylbutyl and 2-ethylhexyl, n-nonyl, decyl, undecyl, dodecyl, tetradecyl, pentadecyl, hexadecyl, heptadecyl, octadecyl; cyclohexyl which can be substituted one to three times with C₁-C₄-alkyl, or C₁-C₄-alkoxy; cyclohexyl condensed by phenyl of the formulae 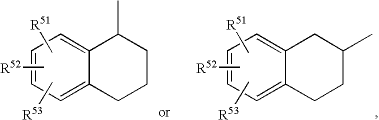
wherein R⁵¹, R⁵² and R⁵³ are independently of each other hydrogen, C₁-C₈-alkyl, C₁-C₈-alkoxy, halogen and cyano; cyclohexenyl, which can be substituted one to three times with C₁-C₄-alkyl, or C₁-C₄-alkoxy; phenyl, biphenylyl, 1- or 2-naphthyl, which can be substituted one to three times by C₁-C₈alkyl, or C₁-C₈alkoxy; or —CR⁷R⁸—(CH₂)ₘ-A³ wherein
R⁷ and R⁸ stand for hydrogen, or C₁-C₄alkyl, A³ stands for phenyl, 1- or 2-naphthyl, which can be substituted one to three times by C₁-C₈alkyl, or C₁-C₈alkoxy; and m stands for 0, or 1.

16. An EL device comprising a composition according to claim 6.

17. Composition comprising
(a) 0.01 to 50% weight, based on the total weight of the colored high molecular weight organic material, of a composition according to any of claim 6,
(b) 99.99 to 50% by weight, based on the total weight of the colored high molecular weight organic material, of a high molecular weight organic material, and
(c) optionally, customary additives in effective amounts.

18. A fluorescent tracer, color changing medium, solid dye laser or EL laser comprising a composition according to claim 6.

19. A composition comprising a guest chromophore and a host chromophore according to claim 6, wherein the host chromophore is a diketopyrrolopyrrole having a photoluminescence emission peak at 520 to 630 nm.

20. A composition comprising a guest chromophore and a host chromophore, according to claim 6, wherein the host chromophore is a diketopyrrolopyrrole having a photoluminescence emission peak at 540 to 600 nm.
